SEQUENCE LISTING (4 sequences)

SEQ ID NO: 1 - DNA, 21 bp, Artificial Sequence
  Synthetic primer
aaaccaaaccacaagacagac

SEQ ID NO: 2 - DNA, 18 bp, Artificial Sequence
  Synthetic primer
gctgaaggcatctcggag

SEQ ID NO: 3 - DNA, 21 bp, Artificial Sequence
  Synthetic primer
ctatggtgagccgtgattgtg

SEQ ID NO: 4 - DNA, 21 bp, Artificial Sequence
  Synthetic primer
tctgtgtcatcctcctgtgtc

(19) United States
(12) Patent Application Publication
(10) Pub. No.: US 2017/0176446 A1
(43) Pub. Date: Jun. 22, 2017

(54) Methods for Determining the Oncogenic Condition of Cell, Uses Thereof, and Methods for Treating Cancer

(71) Applicants: INSERM (INSTITUT NATIONAL DE LA SANTE ET DE LA RECHERCHE MEDICALE), Paris (FR); Affichem, Toulouse (FR)

(72) Inventors: Philippe De Medina, Colomiers (FR); Michael Paillasse, Toulouse (FR); Marc Poirot, L'Union (FR); Sandrine Silvente-Poirot, L'Union (FR)

(21) Appl. No.: 15/404,473

(22) Filed: Jan. 12, 2017

Related U.S. Application Data
(63) Continuation of application No. 14/098,089, filed on Dec. 5, 2013, now abandoned.
Application No. 14/098,089 is a continuation of application No. 13/379,143, filed on Dec. 19, 2011, now abandoned.

(30) Foreign Application Priority Data
Jun. 25, 2009 (FR) 0903101

(51) Int. Cl. G01N 33/574; A61K 31/138; A61K 31/40; A61K 31/58; A61K 31/4535; A61K 31/565; A61K 31/4515; A61K 31/135; A61K 31/55; A61K 31/4015; A61K 31/496; A61K 31/343; A61K 31/5415; A61K 31/5685; A61K 31/195; G01N 33/92

(52) U.S. Cl. CPC G01N 33/57496; G01N 33/92; A61K 31/138; A61K 31/40; A61K 31/58; A61K 31/4535; A61K 31/565; A61K 31/4515; A61K 31/135; A61K 31/55; A61K 31/4015; A61K 31/496; A61K 31/343; A61K 31/5415; A61K 31/5685; A61K 31/195; G01N 2405/00

(57) ABSTRACT

The invention relates to methods for detecting the oncogenic condition of cells, including step where the amount of the OCDO compound in said cells is measured, and to the uses thereof. The invention further relates to OCDO inhibitors for use in methods for treating cancer.

### FIELD OF THE INVENTION

The invention relates to methods for determining the oncogenic state of a cell and to uses thereof. The invention also relates to OCDO inhibitors for use in methods for treating cancers.

### INTRODUCTION

The diagnosis of cancer is based on various elements, such as, in particular, auscultation and medical examinations (endoscopy, fibroscropy, radioscopy, etc.), which allow the physician to note the visible or palpable signs of the disease, but also blood and urine tests which make it possible to determine the number of red blood cells, of white blood cells and of platelets, the hemoglobin level and the creatinine level, or else on the detection of the presence of specific cancer markers in samples taken from the patient. Many cancer markers have been described in the literature. They are generally specific for certain types of cancers in particular, or even subpopulations of patients. Moreover, the markers described in the literature do not generally make it possible to definitely diagnose cancer, and the rates of false-negatives or false-positives are still high. Finally, some markers can be detected only at a late stage in development of the cancer, which at least partly compromises the success of the treatment.

There is therefore a need for new cancer markers which make it possible to diagnose any type of cancer, at an early stage and with certainty and reproducibility.

### SUMMARY OF THE INVENTION

The invention relates to a method for detecting the oncogenic state of cells of a sample obtained from an individual, characterized in that it comprises a step in which the amount of the compound OCDO of formula

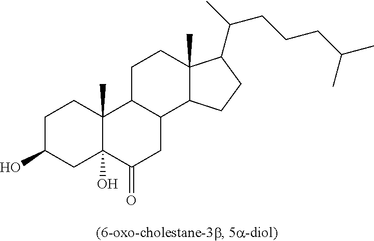 [SMILES: CC(C)CCCC(C)C1CCC2C3CC(=O)[C@@]4(O)C[C@@H](O)CC[C@]4(C)C3CC[C@]12C]

present in the cells taken is determined.

The invention also relates to a method for diagnosing cancer in an individual, characterized in that it comprises a step in which the amount of the compound OCDO, or 6-oxo-cholestane-3β,5α-diol, in the cells of a sample obtained from said individual is determined.

The invention also relates to the use of the compound OCDO as a marker for the oncogenic state of a sample obtained from an individual.

The invention also relates to a method for monitoring the response to a treatment of an individual suffering from cancer, said method comprising the steps of determining, before and during the treatment, the oncogenic state of cells of a sample obtained from said individual or the diagnosis of the individual using the methods according to the invention; a change in the oncogenic state or in the diagnosis of the individual during the treatment being indicative of a response by the individual to said treatment.

The invention also relates to a method for evaluating the efficacy of a medicament for treating a cancer in an individual suffering from said cancer, characterized in that
(a) the concentration D1 of OCDO in a liquid extract of the cells of a sample obtained from said individual is assayed;
(b) after a therapeutic treatment time, the concentration D2 of OCDO in a liquid extract of the cells of a sample obtained from said individual is assayed in the same way as in step (a);
(c) D1 and D2 are compared; and
(d) if D2<D1, it is deduced therefrom that the medicament is effective for treating said cancer.

A subject of the invention is also a method for evaluating the efficacy of a cancer treatment in an individual suffering from said cancer, said method comprising the steps of determining, before and during the treatment, the oncogenic state of cells of a sample obtained from said individual or the diagnosis of the individual using the methods according to the invention; a change in the oncogenic state or in the diagnosis of the individual during the treatment being indicative of the efficacy of said treatment for treating the cancer.

The invention also relates to the OCDO inhibitors for use in a method for treating a cancer in humans or animals.

### DEFINITIONS

For the purpose of the invention, the term “biological material” or “sample” is intended to mean a biological tissue, a preparation or an extract derived from biological tissue, which is liquid or solid; the material may also be a mixture of at least two materials as defined above. Such a sample or biological material can therefore be, in particular, either prepared from tissues, organs, stools or biological fluids from a human or from a mammal, or obtained from cell cultures “in vitro”; such a sample or biological material may also be blood, serum, plasma, urine, cerebrospinal fluid, synovial fluid, peritoneal fluid, pleural fluid, seminal fluid or ascitis fluid. Typically, a sample or biological material according to the invention is a biopsy of a cancerous tissue or a tissue suspected of being cancerous.

For the purpose of the invention, the term “individual” is intended to mean a human or animal mammal.

The term “oncogenic state” of a cell is intended to mean a dedifferentiated state in which the proliferation program of the cell has been modified such that said cell proliferates in an uncontrolled manner, which can lead to the formation of invasive and/or metastatic malignant tumors.

The terms “treatment” and “treating” refer to any act aimed at improving the state of health of an individual, for instance therapy, prevention, prophylaxis or slowing of the disease. In certain embodiments, these terms refer to an improvement in or the eradication of a disease or of symptoms associated with this disease. In other embodiments, these terms refer to a decrease in the progression or in the malignancy of the disease.

The term “therapeutically effective amount” is intended to mean an amount sufficient to treat the individual.

The term “cancer” is intended to mean any type of disease in which certain cells of the human or animal body divide in an uncontrolled manner. The cancers are typically selected from carcinomas, sarcomas and hematopoietic cancers. More particularly, the cancer according to the invention is breast cancer, lung cancer, melanoma, colon cancer, rectal cancer, pancreatic cancer, multiple myeloma, leukemia, lymphoma, Kaposi's sarcoma, testicular cancer, prostate cancer, uterine cancer, glyoma, neuroblastoma, osteosarcoma, embryonic carcinoma or medullary carcinoma of the thyroid.

### DETAILED DESCRIPTION OF THE INVENTION

The present invention relates to the identification of a marker, OCDO, which appears early in tumor cells and which exhibits mytogenic and tumor invasiveness-stimulating properties. The invention is based on the detection of this marker and the assaying thereof in samples obtained from individuals suspected of suffering from or suffering from cancer. The invention also relates to OCDO-inhibiting molecules for use thereof in methods for treating cancers.

Cholesterol and cholesterol oxidation products have for a long time been suspected of having carcinogenic properties in humans (Fritz Bischof, Advances in Lipid Research, vol. 7, p. 165-244, 1969). These effects were initially observed on a limited number of rodent species and resulted in contradictory observations which led to disinterest by the scientific community regarding this subject (Leland L Smith et al., Free Radical Biology and Medicine, vol. 7, p. 285-332, 1989). Certain cholesterol oxidation products are known to appear during the catabolism of cholesterol and the production of steroid hormones. A certain number of oxysterols play a key physiological role in the immune system, the nervous system and the cardiovascular system. The known targets of oxysterols are ligand-dependent transcription factors, the most well known of which are LXR receptors (liver-X-receptor). Oxysterols modulate the intracellular transport of cholesterol, the formation of lipid microdomains, the activation of HedgeHog pathways involved in morphogenesis during embryonic development; they also have antagonistic properties on aromatic hydrocarbon receptors.

It has been indicated that certain oxysterols modulate the activity of enzymes involved in cholesterol metabolism and, in particular, in mevalonate biosynthesis (HMG-CoA-reductase), cholesterol esterification and cholesterol epoxide hydrolysis (see Schoepfer G. Jr.: Physiological Reviews, vol. 80, No. 1, p. 361-554, 2000). It has also been noted that, depending on the nature of the oxygen-bearing group (alcohol, carbonyl, hydroperoxide, peroxide or epoxide), its position on the hydrocarbon backbone of cholesterol, its spatial orientation and the number of oxygen-bearing groups on said hydrocarbon backbone, a cholesterol oxidation product can have specific recognition properties for various biological targets (membranes, enzymes, receptors).

According to the invention, it has been noted that cholestane-3β,5α-diol-6-one (OCDO) is present in varying tumor lines of tissue origin. The properties of this molecule have been characterized in tumor mytogenesis and, in vitro, in invasiveness on cells and on tumors implanted in rodents. It has also been shown that this molecule leads, in tumor cells, to a stimulation of the expression of immunosuppressive cytokines, whereas it decreases the expression of immunostimulatory cytokines, which is in agreement with an immunosuppression in the vicinity of the tumor, promoting its development and its invasiveness.

It is known that cholesterol epoxide hydrolase (ChEH) is an enzyme responsible for the hydrolysis of cholesterol epoxide (CE) to give cholestane triol (CT) according to the following reaction:

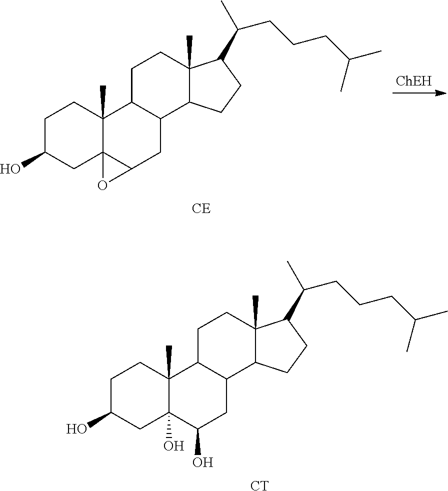 [SMILES: CC(C)CCC[C@H](C)C1CCC2C3CC4OC45C[C@@H](O)CC[C@]5(C)C3CC[C@@]21C ; CC(C)CCC[C@H](C)C1CCC2C3C[C@@H](O)[C@@]4(O)C[C@@H](O)CC[C@]4(C)C3CC[C@@]21C ; "CT"]

(see Médina et al., Faseb J. 19(4): A285-A285, Part I Suppl. S, 4 Mar. 2005).

In a first step, the applicants compared the activity of ChEH for cell extracts obtained from cells of normal tissues and from tumor cells: the activity of ChEH can be observed by separating, by thin-layer chromatography, the CE substrate with respect to the CT product of ChEH. The spots corresponding to the CE and CT compounds were visualized by ¹⁴C radiolabeling. The details of the experiment are provided in assay 1 described in detail later, and the result is represented in FIG. 1.

The applicants noted that the normal cells made it possible to visualize two spots corresponding to the CE and CT compounds, but that, on the other hand, surprisingly, the tumor cells revealed three spots, namely one corresponding to cholestane triol (CT), the second corresponding to cholesterol epoxide (CE) (weak compared with the corresponding spot relative to the normal cells), and a third spot close to that of the CE compound, but nevertheless perfectly distinct.

It is known that tamoxifen (tam), used in a known manner as a tumor-reducing anticancer agent for the treatment and prevention of breast cancers, is an inhibitor of ChEH and that the same is true for PBPE (N,N-pyrrolidino-[(4-benzyl)phenoxy]ethanamine) which is a known antiproliferative agent (in this regard, see the publication reference given in assay 2 of the present application). Neither this “third spot”, which the applicants attributed to a metabolite M, nor the CT spot exists if the tumor cells are treated with tamoxifen. Given that tamoxifen inhibits ChEH, it was investigated, in a second step, whether the inhibition of ChEH could be responsible for the appearance of the “third spot” on the chromatography plate of the treated tumor cells. The details of this experiment are provided in assay 2 described later, and the results are represented in FIGS. 3A and 3B. To do this, an inhibition of ChEH activity was carried out, on extracts of tumor cells treated respectively with tamoxifen or with PBPE, the incubation of the cells with the inhibitor being maintained for three days, with increasing doses of inhibitor for the successive assays. In addition, it was noted, on the thin-layer chromatographs, that, when the dose of inhibitor increases, the CT compound disappears at the same time as the “third spot” in the vicinity of the spot for the CE compound. According to the invention, the applicant deduced therefrom that the metabolite M relating to the “third spot” was probably a derivative of the CT compound.

In a third step, the applicants completed this experiment by establishing that the formation of the metabolite M corresponding to the “third spot” took place gradually, after an incubation of more than 24 hours with the ChEH inhibitor tamoxifen, to the detriment of the formation of the CE compound; and this phenomenon occurs with the two α and β isomers of the CE compound. This further demonstration is detailed in assay 3 and represented in FIGS. 2A and 2B. The applicants therefore concluded therefrom, according to the invention, that the product relating to the “third spot” is a CT-compound conversion product, which has a chromatographic behavior close to that of the CE compound but is retained to a greater extent by the chromatography support, which indicates that the metabolite M associated with the “third spot” is a compound which has an intermediate polarity between those of the CT and CE compounds and which has a structure similar to that of the two CE and CT molecules.

Assay 4 detailed below provides all the information of the study carried out in order to confirm that the metabolite M of the “third spot” originates from a conversion of the CT compound; the result of this assay is represented in FIG. 4.

Finally, assay 5 describes the method which enabled the chemical identification of the product corresponding to this “third spot”. The applicants therefore established, according to the invention, that this “third spot”, which appears only with the tumor cells assayed, is due to 6-oxo-cholestane-3β,5α-diol (OCDO) corresponding to the formula below:

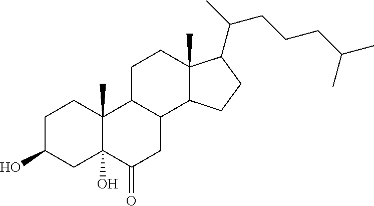 [SMILES: CC(C)CCCC(C)C1CCC2C3CC(=O)[C@@]4(O)C[C@@H](O)CC[C@]4(C)C3CC[C@]12C]

The OCDO product is not a new product: it was described as a product of oxidation of cholestane-3β,5α,6β-triol (CT) by N-bromosuccinimide (Fieser L. F. et al.; Rajagopalan S.: Selective oxidation with N-bromosuccinimide. II. Cholestane-3β,5α,6β-triol, J Am Chem Soc, 71, p. 3938-41, 1949). The chemical synthesis of OCDO had, moreover, already been described in 1908 (Robert Howson Pickard et al., J. Chem. Soc. Trans. vol. 93, p. 1678-1687, 1908).

The existence of this molecule as a metabolite resulting from the conversion of cholestane-3β,5α,6β-triol was reported in 1971: it was indicated that OCDO could be found in the feces of rats force-fed cholestane-3(3β,5Δ,6β-triol (Roscoe H G, Fahrenbach M J, J Lipid Res, vol. 12, p. 17-23, 1971). It was also indicated that OCDO could be found in bovine serum and human blood at a concentration of between 10 and 100 nM (Yamaguchi M. et al., Biol. Pharm. Bull. 20(9), p. 1044-46, 1997).

The detailed experimental procedure implemented for assays 1 to 5, which made it possible to result in the identification of the OCDO marker, will be given hereinafter.

For assays 1 to 5 and in some of the examples given later in this patent application, MCF7 tumor cells, which originate from the American Tissue Culture Collection (ATCC), were used. These cells are cultured in RPMI 1640 medium supplemented with 2 g/liter of aqueous sodium carbonate, 1.2 mM of glutamine (pH 7.4 at 23° C.) and 5% of fetal bovine serum (Gibco), at 37° C. under 5% CO₂, and 2.5 ml of antibiotics (penicillin/streptomycin) per liter of medium. It is desired to study the activity of ChEH in mouse cells by silica thin-layer chromatography. The compounds that it is desired to visualize are the CE and CT compounds defined above. In order to visualize the spots corresponding to CE and CT on a chromatography plate, ¹⁴C-labeled CE and CT compounds were synthesized.

### a) Synthesis of [¹⁴C]5,6-β-epoxycholestan-3β-ol and [¹⁴C]5,6-α-epoxycholestan-3β-ol

0.35 μmol of [¹⁴C]cholesterol (58 mCi/mmol) is dissolved in 200 μl of dichloromethane in the presence of 0.56 μmol of meta-chloroperbenzoic acid. The solution is stirred at ambient temperature for 5 hours. The reaction mixture is dissolved in 1 ml of dichloromethane, and washed with aqueous sodium sulfite (10% by weight), sodium hydrogen carbonate (aqueous solution at 5% by weight) and a saturated solution of sodium chloride. The organic phase is evaporated off and the residue is purified by RP-HPLC (Ultrasep ES C18 6 μm hydrophobic column) under CH₃OH/H₂O (95/5 by volume) isocratic conditions, at 0.7 ml/min. The α and β isomers are readily separated under these conditions and detected with a radioactivity detector (Berthold).

The total yield from the reaction is 80%: the product obtained contains 75% of α isomer (CEα) and 25% 5 of 3 isomer (CEβ).

### b) Synthesis of [¹⁴C]cholestane-3β,5α,6β-triol

This compound was synthesized from [¹⁴C]5,6-β-epoxycholestan-3β-ol as described in the literature (Pulfer M K and Murphy R C, Formation of biologically active oxysterols during ozonolysis of cholesterol present in lung surfactant, J Biol Chem, vol. 279(25), p. 26331-26338, 2004). The [¹⁴C]CEβ prepared in a) above (58 mCi/mmol) is dissolved in 1 ml of a tetrahydrofuran/H₂O/acetone mixture (v/v/v, 4:1:0.5). 125 μl of perchloric acid are added to the reaction medium, which is stirred for 4 hours at ambient temperature.

The reaction mixture is diluted in 1 ml of dichloromethane and then washed with sodium hydrogen carbonate (aqueous solution at 5% by weight) and with water. The residue is purified by HPLC on a hydrophobic column (Ultrasep ES C18 6 μm) under CH₃OH/H₂O (95/5 by volume) isocratic conditions, at a flow rate of 0.7 ml/min. [¹⁴C]CT is obtained with a yield of 62%.

In order to be used in assays 1 to 5 which follow, the cells are seeded into 6-well plates at a density of 80 000 cells in a volume of 2 ml. Thirty-six hours after seeding, the cells are treated for 15 minutes either with vehicle solvent (ethanol at 1 in a PBS buffer) or with the compounds that it is desired to test; this incubation is therefore, as required by the assay, carried out with the [¹⁴C]CEα (0.6 μM, 15 μCi/μmol) and [¹⁴C]CEβ (0.6 μM, 15 μCi/μmol) or [¹⁴C]CT (1 μM, 15 μCi/μmol) compounds; the proportion of solvent does not exceed 20 relative to the volume of the culture medium. After the incubation time desired for the assay, the medium is collected, the cells are washed with cold PBS (phosphate buffer) (2 ml per well) which is pooled with the medium. The cells are then scraped into cold PBS (1 ml for 3 wells); the wells are again rinsed with cold PBS (1 ml for 3 wells). The cell suspension obtained is centrifuged at 1000 rpm for 5 min at 4° C. The cell pellet and the medium are extracted by the modified Folch method (as published by Ways P. et al., J Lipid Res, 5(3): 318 (1964)). Throughout the rest of this description, the vehicle solvent used is the same as that defined above.

The aqueous and organic radioactivities are counted. The organic phases are brought to dryness under argon. The residue is resuspended in 60 μl of ethanol and then deposited, in a proportion of 20 μl per lane, on to the glass-backed silica plates (Whatman LK-6-DF, 20×20), which are used in the various assays (these plates having been preheated at 100° C. for 1 hour). The migration solvent used is ethyl acetate. The chromatography plates are placed in contact with a “phosphor-screen” plate in a cassette overnight. The “phosphor-screen” is revealed with a “PhosorImager” of Storm type. In order to evaluate the more or less dense nature of the spots obtained on the plates, the radioactivity is quantified by densitometry with the “Imagequant”™ computer program.

### Assay 1

In this assay 1, the activity of ChEH was demonstrated in healthy mouse cells and in MCF7 mouse tumor cells. The process is carried out by thin-layer chromatography according to the techniques which have just been described. The results are given in FIG. 1.

In this figure, it is seen that all the deposits were made at the same level marked by a dot-dashed line at the bottom of the lanes.

The [¹⁴C]CE compound prepared beforehand as indicated above (0.6 μM, 15 μCi/μmol) was deposited on the left lane. The middle and right lanes correspond to extracts of cells incubated beforehand in [¹⁴C]CE according to the technique described above.

The cell extract deposited at the bottom of the right lane is an extract corresponding to normal hepatocytes taken from adult C57/B16 mice weighing 20-25 g (supplied by Charles River). The hepatocytes were isolated by perfusion with collagenase according to the protocol by Davis (Davis R A et al., J. Biol Chem, 1979, vol. 254, No. 6, p. 2010-2016) and cultured in collagen-coated Petri dishes 6 cm in diameter, at a density of 2 million cells per dish, in nutritive Dulbecco's modified eagle medium (DMEM) containing 10% of fetal calf serum, insulin (0.5 U/ml) and antibiotics (50 units/ml) (a mixture of penicillin and streptomycin). The dishes are kept at 37° C. in a humid incubator with 5% CO₂. After adhesion of the cells, the nutritive medium is replaced with new medium after having washed the cells with PBS buffer so as to remove the cell debris. The cells are used as early as the following day for the assay.

It is seen, on the right lane of FIG. 1, that the CT compound appears first and that the CE compound is substantially at the level of that which was deposited directly on the left lane. The CT compound formed necessarily originates from the CE compound converted by the ChEH hydrolase, since both appear on the chromatography, although, initially, only the labeled CE compound was deposited and capable of appearing.

On the other hand, on the middle lane, extracts of MCF7 mouse tumor cells were deposited; this cell extract is prepared from MCF7 cells cultured in the same way as the extract of hepatocytes from the C57/B16 mouse cells. A spot corresponding to the CT compound is noted, and then two spots, close to one another, one corresponding to the CE compound of the right lane and the other to an unidentified metabolite M; in addition, this “third spot” is more intense than that corresponding to the CE compound.

It was thus deduced that, in the tumor cells, a part of the CE compound had been converted into a metabolite M having a chromatographic behavior close to that of the CE compound and having an intermediate polarity between those of the CE and CT compounds.

### Assay 2

It was investigated whether or not the appearance of the “third spot” on the chromatography plate of assay 1 was affected by inhibition of ChEH, which, in the cell, gives rise to the CT compound from the CE compound. It is known that ChEH is inhibited by tamoxifen (Tam) and by PBPE (FASEB Journal, vol. 19, Issue 4, p. A285-A285, Part 1 Suppl. S). These two inhibitors were thus used for this assay.

To do this, cell extracts of MCF7 tumor cells incubated in a solution of [¹⁴C]CE (-α or -β) as indicated in assay 1, and then subsequently incubated in an aqueous solution of ChEH inhibitor, namely tamoxifen or PBPE, for 3 days, were deposited on to a chromatography plate of the same type as that used for assay 1. When an incubation with [¹⁴C]CE-α is used, tamoxifen solutions at concentrations of 1×10⁻², 1×10⁻¹, 5×10⁻¹, 1, 2.5 and 5 μM are employed (FIG. 3A); when an incubation with [¹⁴C]CEβ is used, PBPE solutions at concentrations of 1×10⁻², 1×10⁻¹, 1, 5 and 10 μM are employed (FIG. 3B); FIGS. 3A and 3B represent the chromatographic plates obtained in the two cases. It is noted that, for the high amounts of inhibitor used in the incubation, the CT compound and the “third spot” due to the metabolite M simultaneously disappear; conversely, for the low amounts of inhibitor, both the spot of the CT compound and the spot due to the metabolite M are substantial, whereas the spots due to the CE are weak, which shows that the CE has been converted into (CT+ metabolite); it is concluded therefrom that the ChEH hydrolase, when it is relatively noninhibited, enables the CT to appear and, consequently, the spot of the metabolite M also appears.

### Assay 3

The kinetics of the activity of ChEH in MCF7 cells were studied using the [¹⁴C]-labeled CE (-α or -β) compound. As in assay 2, the cells are incubated with [¹⁴C]CE (-α or -β). The extracts are deposited on the chromatography plates after incubation times of 4, 8, 16, 24, 48 and 72 hours: the plates obtained are represented in FIGS. 2A (for CEα) and 2B (for CE). The deposits, as for FIGS. 1 and 3A and 3B, were made at the same level marked by a dot-dashed line at the bottom of the lanes. For a short period of incubation, it is seen that the CE compound does not have time to be converted a great deal into CT compound; however, if the incubation time increases, the ChEH increasingly converts the CE compound into CT compound, such that the CT spots are more dense whereas the CE spots become lighter; and simultaneously, when the CT compound appears, the “third spots” corresponding to a metabolite M are seen to appear.

The applicant therefore considered it to be likely that the metabolite M of the “third spot” was a derivative of the CT compound.

### Assay 4

In order to verify the conclusions drawn from examples 1 to 3, MCF7 cells were incubated with the [¹⁴C]CT compound for periods of 24, 48 and 15 72 hours using the procedures defined above. The cell extracts were subsequently obtained as indicated in assay 1 and deposited on a chromatography plate (see FIG. 4). The left lane of the plate receives a deposit of [¹⁴C]CE and the neighboring lane receives a deposit of [¹⁴C]CT, as migration controls; the other three lanes correspond to the cell extracts assayed after incubation. It is noted that, for an incubation for a period of 24 hours, a spot corresponding to that of the metabolite is seen in the vicinity of the spot of the [¹⁴C]CE compound, on the lane of the cell extract. The longer the incubation time, the more dense this spot is, and the darker the spot of the metabolite M is, the lighter the spot of the CT compound becomes.

This confirms that the metabolite is indeed a CT-compound conversion product.

### Assay 5

In order to identify the chemical structure of the metabolite that appeared in assays 1 to 4, a multistep technique was used. MCF7 cells were seeded at 0.4×10⁶ cells per Petri dish (100 mm diameter) in 10 ml of the medium defined in assay 1. Thirty-six hours after seeding, some of the cells were incubated, at a concentration of 10 μM, in CEα and the others were incubated, at a concentration of 10 μM, in [¹⁴C]CE obtained as indicated above. After seventy-two hours, the cells are washed with cold PBS (phosphate buffer), and then scraped into cold PBS and centrifuged at 1000 rpm for 5 minutes at 4° C. The cell pellet and the medium are extracted by the modified Folch method (see the reference already provided on page 9 of the present text).

The organic phase is evaporated, resuspended in methanol and then passed through an RP C18 cartridge (Sep-Pack from the company Waters). The cartridge is then washed with methanol. After evaporation, the residue is dissolved in 20 μl of ethanol and then purified by reverse-phase HPLC (“Ultrasep” hydrophobic column) using isocratic conditions: CH₃OH/H₂O (95/5 by volume) at 0.7 ml/min. 1 mn fractions are collected at the column outlet and the radioactivity is counted in order to determine the retention times of the labeled compounds and in particular of the metabolite resulting from the [¹⁴C]CEα. The radioactive fractions are analyzed by thin-layer chromatography using ethyl acetate as migration solvent. The HPLC fractions of interest resulting from the MCF7 cells treated with cold CEα are analyzed by electron impact (70 ev) and chemical ionization mass spectrography (see the spectrum in FIG. 5).

It was thus determined that the CT produced by the MCF7 cells has a mass of 420 and a chromatographic behavior similar to that of the commercial CT. The mass spectrometry of the metabolite existing in the MCF7 cell extracts and purified as indicated above gives a mass of 418, i.e. a loss of 2 mass units relative to the CT compound. As is seen from assays 1 to 4, the metabolite originates from the bioconversion of the CT compound: the difference in mass therefore corresponds to a loss of 2 hydrogen atoms. This suggests the appearance of a ketone function or the appearance of a double bond on the CT compound. The infrared analysis shows the appearance of a band characteristic of a ketone function, which demonstrates the formation of 6-oxo-cholestane-3β,5α-diol (OCDO). The structure of the OCDO compound corresponds to a product from dehydrogenation of the CT compound on the hydroxyl group carried by carbon 6 of the nucleus. The chromatographic properties and the fragmentation profile in mass spectrometry of the metabolite are identical to those of the commercial standard supplied by the company Steraloids for the OCDO compound, which demonstrates the identity between these two molecules.

### Methods for Diagnosing Cancer

The invention therefore relates to a method for detecting the oncogenic state of cells taken from a sample obtained from an individual, characterized in that it comprises a step in which the amount of the OCDO compound of formula

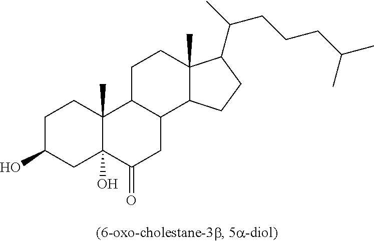 [SMILES: CC(C)CCCC(C)C1CCC2C3CC(=O)[C@@]4(O)C[C@@H](O)CC[C@]4(C)C3CC[C@]12C]

present in the cells taken is determined.

The invention also relates to a method of diagnosis for detecting the oncogenic state of cells taken from a biological material originating from a human individual or from a mammalian animal, characterized in that it is determined, via a visualizing means, whether the cells taken contain the OCDO compound in a significant amount, and that, if this determination is positive, it is deduced therefrom that said cells are in an oncogenic state.

The invention also relates to a method for diagnosing cancer in an individual, characterized in that it comprises a step in which the amount of the OCDO compound in the cells of a sample obtained from said individual is determined.

According to the invention, the amount of OCDO in the cells of the sample from the individual tested is compared with a reference value, said reference value being measured in the cells of a sample from a healthy individual under the same experimental conditions as for the measurement of the amount of OCDO of the cells of the sample from the individual tested. An amount of OCDO that is significantly enhanced compared with the reference value is then indicative of an oncogenic state of the cells of said sample. The term “significantly enhanced” is intended to mean a value statistically greater than the reference value (p<0.05).

The methods according to the invention make it possible typically to provide a prognosis for the progression of a tumor in an individual, at an early stage of the progression of the disease. If the cells of the sample from the individual exhibit an amount of OCDO less than or equal to a reference value, this is indicative of a good prognosis and of a benign tumor. Conversely, if the cells of the sample from the individual exhibit an amount of OCDO greater than a reference value, this is indicative of a poor prognosis and of a malignant tumor.

Typically, the amount of OCDO is determined using a visualizing means.

In one embodiment of the invention, the amount of the OCDO compound is determined (measured) in a liquid extract of said cells. Typically, this liquid extract is obtained by lysing the cells and then separating the solid and liquid fractions, for example by centrifugation. The liquid fraction constitutes said “liquid extract” of cells.

The presence of OCDO in the liquid extract can be found by thin-layer chromatography, the presence of OCDO then being detected on the chromatography plate via an appropriate visualizing means. It is possible to provide for the visualizing means to consist of a chemical modification of the OCDO which allows it to become immobilized on a transport protein, which is subsequently detected with monoclonal antibodies. According to one variant, the visualizing means is radioactive labeling of the cells taken, carried out before the chromatography, the visualization taking place on the plate by quantification of the radioactivity.

It was noted that the cells have an oncogenic state as long as a liquid extract of said cells has an OCDO concentration greater than 1 μM. The OCDO concentration in the extract can be assayed by high-performance liquid chromatography (HPLC). This concentration can also be assayed by gas chromatography followed by mass spectrography.

In one particular embodiment, the amount of OCDO in the liquid extract is determined by thin-layer chromatography, the OCDO compound being detected on the chromatography plate by an appropriate visualizing means.

A subject of the invention is also the use of the OCDO compound as a marker for the oncogenic state of a sample obtained from an individual.

The invention also relates to the use of the OCDO compound as a marker for the oncogenic state of a biological material originating from a human individual or from a mammalian animal.

The invention also relates to the use of the OCDO compound as a marker for the oncogenic state of the cells of a sample obtained from an individual.

The invention also relates to the OCDO compound for use in a method for diagnosing cancer performed on the human or animal body.

Methods for Monitoring the Treatment of an Individual Suffering from Cancer

The invention also relates to a method for monitoring the response to a treatment of an individual suffering from cancer, said method comprising the steps of determining the diagnosis of the individual before and during the treatment using the method of diagnosis according to the invention, a change in diagnosis of the individual during the treatment being indicative of a response by the individual to said treatment.

The invention also relates to a method for monitoring the response to a treatment of an individual suffering from cancer, said method comprising the steps of determining the oncogenic state of cells of a sample obtained from said individual before and during the treatment using the method for detecting the oncogenic state of cells according to the invention, a change in the oncogenic state of the cells of the sample from the individual during the treatment being indicative of a response by the individual to said treatment.

According to the invention, the samples taken before and during the treatment are taken under the same experimental conditions.

### Methods for Screening Anticancer Medicaments

A subject of the invention is also a method for evaluating the efficacy of a medicament for treating a cancer in an individual suffering from said cancer, characterized in that
(a) the concentration D1 of OCDO in a liquid extract of the cells of a sample obtained from said individual is assayed;
(b) after a therapeutic treatment time, the concentration D2 of OCDO in a liquid extract of the cells of a sample obtained from said individual is assayed in the same way as in step (a);
(c) D1 and D2 are compared; and
(d) if D2<D1, it is deduced therefrom that the medicament is effective for treating said cancer.

The invention also relates to a method for evaluating the efficacy of a cancer treatment in an individual suffering from said cancer, said method comprising the steps of determining the diagnosis of the individual before and during the treatment using the method of diagnosis according to the invention, a change in diagnosis of the individual during the treatment being indicative of the effectiveness of said treatment for treating the cancer.

The invention also relates to a method for evaluating the efficacy of a cancer treatment in an individual suffering from said cancer, said method comprising the steps of determining the oncogenic state of cells of a sample obtained from said individual before and during the treatment using the method for detecting the oncogenic state of cells according to the invention, a change in the oncogenic state of the cells of the sample from the individual during the treatment being indicative of the effectiveness of said treatment for treating the cancer.

According to the invention, the samples taken before and during the treatment are taken under the same experimental conditions.

### Methods for Treating Cancer

A subject of the invention is also an OCDO inhibitor for use in a method for treating cancer in humans or animals.

The invention also relates to a method for treating an individual suffering from cancer, said method comprising the administration to said individual of a therapeutically effective amount of an OCDO inhibitor.

According to one embodiment, the OCDO is dissolved in ethanol, the solution obtained is then diluted (typically to 1/1000) in a buffer, in particular a phosphate buffer, and the resulting diluted solution is injected into the individual, typically intratumorally. Depending on the inhibitor used, daily doses of between 1 μg/kg and 500 μg/kg can be injected for a period of time of between 1 and 10 weeks. However, those skilled in the art are able to adjust both the form in which the OCDO inhibitor is administered and the dosage and duration of the treatment, according to the patient and the disease treated.

The OCDO inhibitor can typically be selected from the following products:
inhibitors of an enzyme or enzymes involved in cholesterol biosynthesis, such as lovastatin, Ro 48-8071 inhibitor, U18666A, AY-9944, triparanol, terbinafine and SKF-525A;
cytochrome P 450 inhibitors, lipoxygenases and antioxidants which are active on cholesterol epoxidation, such as ketoconazole and vitamin E;
inhibitors of cholesterol epoxide hydrolase (ChEH) activity, such as PBPE, PCPE, tesmilifene, dendrogenin A (DDA), tamoxifen, 4-hydroxytamoxifen, raloxifen, nitromiphene, clomiphene, RU 39411, BD-1008, haloperidol, SR 31747A, ibogaine, AC-915, rimcazole, amiodarone, trifluoroperazine, U18666A, AY-9944, triparanol, terbinafine and SKF-525A;
the inhibitors selected from the group consisting of:
   estrogen receptor antagonists;
   anti-estrogen membrane binding site (AEBS) ligands;
   ligands of σ-1 and -2 receptors and certain aminoalkyl sterols;
   intracellular cholesterol transport inhibitors; and
   enzyme inhibitors selected from the group consisting of progesterone and Ahr receptor antagonists.

Other aspects and advantages of the present invention are described in the following figures and examples, which should be considered as illustrations which do not limit the scope of the invention.

### BRIEF DESCRIPTION OF THE FIGURES

FIG. 1: Measurement of the ChEH activity in MCF7 cells and in mouse hepatocytes. The cells are incubated in the presence of [¹⁴C]CE and then the lipids are extracted and the sterols are analyzed and separated by silica plate thin-layer chromatography. The chromatography plate is visualized by autoradiography. CE: 5,6-epoxycholestanol, CT: cholestane-3β,5α,6β-triol, M: metabolite M.

FIG. 2: Kinetics of the ChEH activity in MCF7 cells. The MCF7 cells are incubated in the presence of [¹⁴C]CEα (A) or of [¹⁴C]CEβ (B) for increasing times of 4 to 72 hours. The lipids are extracted from the cells and the sterols are analyzed and separated by silica plate thin-layer chromatography. The chromatography plate is visualized by autoradiography. CE: 5,6-epoxycholestanol, CT: cholestane-3β,5α,6β-triol, M: metabolite M.

FIG. 3: Dose-dependent inhibition of the ChEH activity (3 days) in MCF7 cells by tamoxifen and by PBPE. The MCF7 cells are incubated in the presence of [¹⁴C]CEα (A) or of [¹⁴C]CEβ (B) for three days in the presence of increasing concentrations of tamoxifen (A) or of PBPE (B). The lipids are extracted from the cells and the sterols are analyzed and separated by silica plate thin-layer chromatography. The chromatography plate is visualized by autoradiography. CE: 5,6-epoxycholestanol, CT: cholestane-3β,5α,6β-triol, M: metabolite M.

FIG. 4: Conversion of CT into metabolite M. The MCF7 cells are incubated in the presence of [¹⁴C]CT (A) for 24, 48 and 72 hours. The lipids are extracted from the cells and the sterols are analyzed and separated by silica plate thin-layer chromatography. The chromatography plate is visualized by autoradiography. CE: 5,6-epoxycholestanol, CT: cholestane-3β,5α,6β-triol. M: metabolite M.

FIG. 5: Mass spectrum of the metabolite C. The MCF7 cells are incubated in the presence of CE and then the lipids are extracted and then purified by HPLC and the fraction corresponding to the OCDO is analyzed by mass spectrometry.

FIG. 6: Analysis of OCDO production in healthy tissues. The cells originating from various mouse organs are incubated in the presence of [¹⁴C]CT (A) for 48 hours in the presence or absence of tamoxifen. The lipids are extracted from the cells and the sterols are analyzed and separated by silica plate thin-layer chromatography. The chromatography plate is visualized by autoradiography. CT: cholestane-3β,5α,6β-triol. M: metabolite M.

FIG. 7: Effect of OCDO on cell proliferation in vitro. The cells of a human medullary thyroid carcinoma (TT cells) are cultured in the presence of increasing concentrations of OCDO ranging from 0.1 to 5 μM, for 4 days. After this incubation time, the amount of cells is measured and the proliferative factor is determined.

FIG. 8: Effect of OCDO on tumor progression in vivo. TT cells are implanted on nude mice. The mice are treated either with the vehicle solvent (Solvent) or with 50 μg/kg of OCDO by injection once a day, 5 days out of 7, for several weeks. The tumor volume is measured and reported on the graph as a function of time.

FIG. 9: Analysis of lymph nodes. The presence of tumor cells is investigated in the lymph nodes and compared between animals treated with the vehicle solvent (Solvent) or OCDO.

FIG. 10: Histomorphological analysis of the tumors. The tumors of the animals treated with the vehicle solvent (Solvent) or the OCDO are fixed and incubated in the presence of antibodies directed against a cytokeratin, calcitonin and an epithelial membrane antigen (EMA). The visualization is carried out by means of a streptavin-biotin-peroxidase complex followed by incubation with a diaminobenzidine solution, and then staining with hematoxylin is carried out. The presence of brown staining reveals the expression of the proteins labeled with the antibodies.

FIG. 11: Effect of OCDO on cytokine expression. THP1 cells are treated in vitro with 10 μM of OCDO for 4 hours. The total RNAs of the cells are extracted and the expression of the genes encoding IL12 and IL10 is determined and reported on the figure.

FIG. 12: Analysis of OCDO by high performance liquid chromatography (HPLC). The OCDO is separated from the other oxysterols, such as 5,6-epoxycholestanol (CE), cholestane-3β,5αa,6β-triol (CT) and 7-ketocholesterol (7-keto-Ch). UV detection at 210 nm is used.

FIG. 13: Calibration curve for OCDO by HPLC. Increasing amounts of from 0.1 to 5 μg of OCDO are injected and analyzed by HPLC. The area of the peaks corresponding to OCDO is reported as a function of the mass of product injected. A correlation straight line is established from the graph.

FIG. 14: Analysis of the OCDO by gas chromatography (GC). A mixture of 5α-cholestane and OCDO is trimethylsilylated and then analyzed by gas chromatography coupled to mass spectrometry (GC/MS). 3 peaks are obtained. The first (10.62 minutes) corresponds to 5α-cholestane, the second (32.43 minutes) corresponds to OCDO having lost one molecule of H₂O (OCDO-H₂O), the third (36.68 minutes) corresponds to OCDO.

FIG. 15: Mass analyses of the peak emerging at 32.43 minutes by GC. The mass fragmentation profile reveals an OCDO dehydration product (M+472.3). The structure of the product obtained is represented.

FIG. 16: Mass analyses of the peak emerging at 36.68 minutes by GC. The mass fragmentation profile reveals an OCDO dehydration product (M+(—CH₃)546). The structure of the product obtained is represented.

FIG. 17: Calibration curve for OCDO by GC/MS. Increasing amounts of from 6.25 ng to 100 ng of OCDO are injected and analyzed by GC/MS. The area of the peaks corresponding to OCDO is reported as a function of the mass of product injected. A correlation straight line is established from the graph.

FIG. 18: Inhibition of tumor growth by tamoxifen in vivo. Mammary cells of TS/A type are implanted in BALB/c mice. The mice are treated with tamoxifen (Tamoxifen) or with the vehicle solvent (vehicle). The volume of the tumors is measured over time.

FIG. 19: GC analysis of the OCDO content of the tumors. The tumors of the animals treated or not treated with tamoxifen are extracted. The extracts are analyzed by GC/MS. The GC profile of the extracts of the animals treated with the vehicle solvent (A) reveals the presence of peaks corresponding to the OCDO and to its dehydration product (OCDO-H₂O).

FIG. 20: MS analysis of the GC peaks corresponding to OCDO and OCDO-H₂O. The mass analysis confirms the structure of the compounds present in the peaks obtained by GC.

FIG. 21: Inhibition of OCDO production in MCF7 cells by ChEH inhibitors. The MCF7 cells are incubated in the presence of [¹⁴C]CEα for three days in the presence of the vehicle solvent (T), of cholesterol epoxide (CE), of tamoxifen (Tam), of PBPE (PBPE), of raloxifene (Ral), and of tesmilifene (DPPE). The lane marked CEα corresponds to a deposit of [¹⁴C]CEα used as migration standard. The lipids are extracted from the cells and the sterols are analyzed and separated by silica plate thin-layer chromatography. The chromatography plate is visualized by autoradiography. CE: 5,6-epoxycholestanol, CT: cholestane-3β,5α,6β-triol.

FIG. 22: Inhibition of OCDO production in MCF7 cells by ketoconazole, a general cytochrome P450 inhibitor. The MCF7 cells are incubated in the presence of [¹⁴C]CEα for three days in the presence of the vehicle solvent (control) or of 10 μM of ketoconazole. The lipids are extracted from the cells and the sterols are analyzed and separated by silica plate thin-layer chromatography. The chromatography plate is visualized by autoradiography and the amount of CE and of OCDO is quantified.

FIG. 23: Inhibition of OCDO production in MCF7 cells by an aminoalkyl sterol (DDA). Lane 1 corresponds to a deposit of [¹⁴C]CEα used as migration standard. Lane 2 corresponds to a deposit of [¹⁴C]CTα used as migration standard. The MCF7 cells are incubated in the presence of [¹⁴C]CEα for 48 hours in the presence of the vehicle solvent (lane 3), of 0.1 μM of DDA (lane 4) and of 1 μM of DDA. The lipids are extracted from the cells and the sterols are analyzed and separated by silica plate thin-layer chromatography. The chromatography plate is visualized by autoradiography. CE: 5,6-epoxycholestanol, CT: cholestane-3β,5α,6β-triol.

FIG. 24: Inhibition of OCDO production in MCF7 cells by intracellular cholesterol transport modulators and AhR receptor modulators: the MCF7 cells are incubated in the presence of [¹⁴C]CTα for 24 hours in the presence of the vehicle solvent (lane 1), of progesterone (lane 2), of U18666A (lane 3), of TCDD (lane 4), of benzo(A)pyrene (lane 5), of resveratrol (lane 6) and of PDM2 (lane 7). The lipids are extracted from the cells and the sterols are analyzed and separated by silica plate thin-layer chromatography. The chromatography plate is visualized by autoradiography. CT: cholestane-3β,5α,6β-triol.

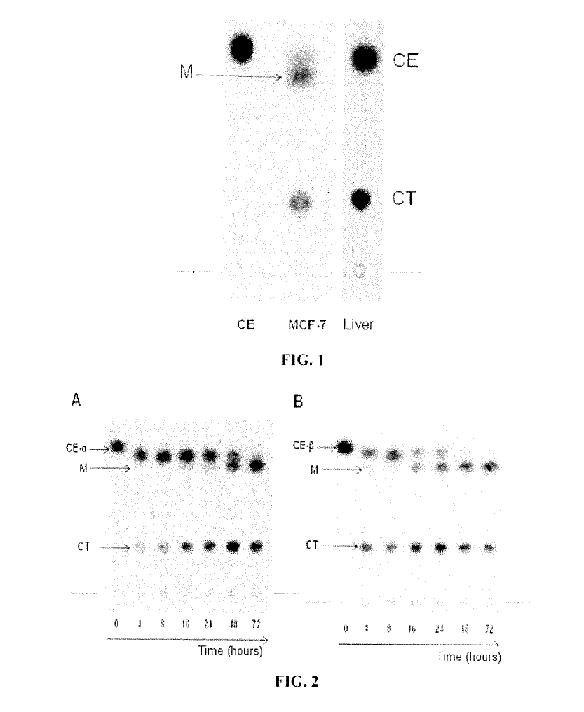

### EXAMPLE 1: OCDO IS A MARKER FOR THE TUMOR STATE OF A CELL

Using the method described in example 4 hereinafter, the presence of OCDO was tested on numerous tumor cell lines in order to establish that this marker can be observed in various cell types in humans, rats or mice. OCDO production was detected in each of the tumor cell lines tested, as indicated in the following table:

| | | OCDO |
|---|---|---|
| Cell line | Type | production |
| MCF7 | Human mammary carcinoma | + |
| MCF7/TamR | Tamoxifen-resistant human mammary | + |
| | carcinoma | |
| MDA-MB-231 | Human mammary carcinoma | + |
| TS/A | Murine mammary carcinoma | + |
| A-549 | Human lung carcinoma | + |
| B16-F10 | Murine melanoma | + |
| SK-MEL-28 | Human melanoma | + |
| U-937 | Human myeloid leukemia | + |
| J 774 | Murine myeloid leukemia | + |
| THP.1 | Human acute monocytic leukemia | + |
| HT-29 | Human colon carcinoma | + |
| HeLa | Human uterine carcinoma | + |
| C6 | Rat glyoma | + |
| SK-N-SH | Human neuroblastoma | + |
| Saos-2 | Human osteosarcoma | + |
| P19 | Murine embryonic carcinoma | + |
| TT | Human medullary thyroid carcinoma | + |

Having noted that OCDO is found in all the tumor cells tested, the applicants concluded that OCDO constituted a marker for the tumor state of the cells.

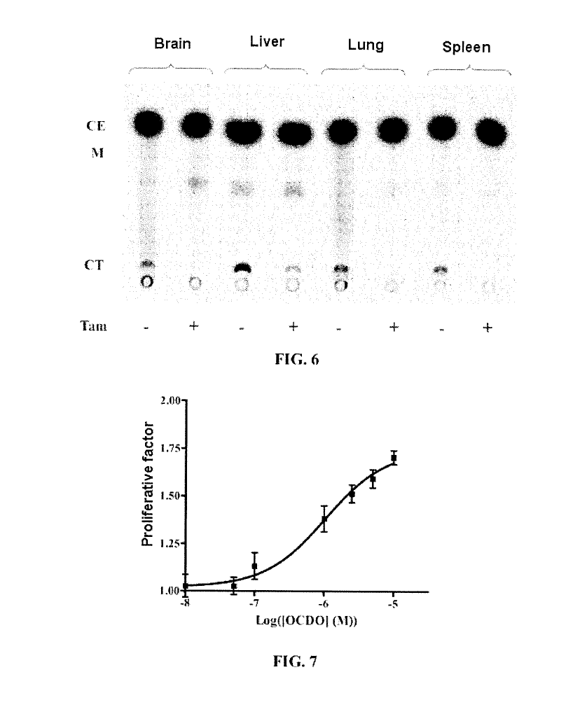

However, as a preliminary study, and in a manner analogous to what was done in assays 1 to 5 above, the conversion of the CE compound was evaluated in cells of various healthy mouse tissues under conditions which made it possible to detect OCDO (see the result in FIG. 6). Said cells were incubated with the [¹⁴C]CEα compound and the radioactive spots which appear with (+) or without (−) incubation with tamoxifen were observed by silica thin-layer chromatography. It is noted that, if tamoxifen is present, the CT compound no longer appears, which corresponds well to the inhibition of ChEH; in the absence of incubation with tamoxifen, the CT compound appears, but no presence, in the vicinity of the CE compound, of a spot corresponding to the presence of OCDO is noted.

It is therefore clear that OCDO is not produced in a normal tissue; its detection therefore clearly constitutes an element for predicting the tumor state of the cells; OCDO is a marker for the oncogenic state of a cell.

### EXAMPLE 2: OCDO IS CARCINOGENIC

On the basis of the assays described above and of example 1, the applicants thought, according to the invention, that the presence of OCDO in the cells in a tumor situation could be linked to the fact that this molecule could by itself have a carcinogenic potential.

Such a potential has never been reported previously in the literature.

Cholesterol does not have mutagenic properties. The OCDO precursors and the cholesterol epoxide epimers have been mentioned as weak mutagens in mammalian cells (Peterson A R, Peterson H, Spears C P, Trosko J E and Sevanian, Mutation Research, vol. 203, p. 355-366, 1988). Sterol epoxides have not shown any mutagenic properties in the Ames test carried out on bacteria (Smith L L et al., Mutation Research, vol. 68, p. 23-30, 1979; Ansari GAS et al., vol. 20, p. 35-41, 1982). These observations were recently confirmed (Cheng Y W et al., Food and Chemical Toxicology, vol. 43, p. 617-622, 2005). Admittedly, structural homologies of OCDO with tumor promoters such as TPA (12-tetradecanoylphorbol 13-acetate) (Endo Y. et al., Chem. Pharm. Bull. (Tokyo), vol. 42 (3), p. 462-469, 1994) have been found. However, no-one has described or even suggested that OCDO could have PKC-activating properties or tumor-promoting properties in laboratory animals; it has only been shown that this molecule binds to a phorbol ester-binding protein (Endo Y., Biochem. Biophys. Res. Comm., vol. 194, p. 1529-1535, 1993).

From the cellular point of view, it has only been indicated that OCDO appears:
1) to inhibit rosette formation by T lymphocytes originating from human serum (Streuli R. A. et al., J. Immunol., vol. 123 (6)n, p. 2897-2902);
2) to inhibit leukocyte mobility (Gordon L., Proc. Natl. Acad. Sci. USA, vol. 77(7), p. 4313-4316, 1980);
3) to induce NK cell toxicity in mice (Kucuk O. et al., Cell. Immunol., 139, p. 541-549, 1992);
4) to inhibit the cytolytic activity produced by T lymphocytes in mice (Kucuk O et al., Lipids, vol. 29(9), p. 657-660, 1994), these effects being observed at concentrations between 1 and 20 μM.

Through the present example 2, the applicants established, in the context of the present invention, that OCDO has effects on tumor development, which were not in any way suggested to those skilled in the art by the prior art.

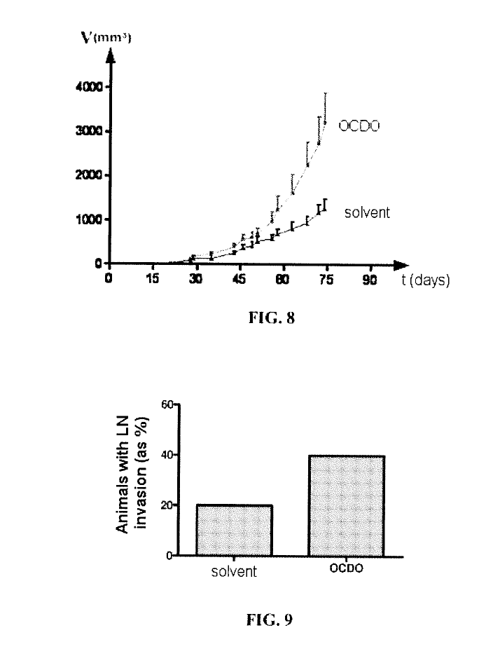

a) In this example 2, the human medullary thyroid carcinoma cell line TT (American Tissues and Cells Collection) was used. This line is cultured in an F12K medium modified by Kaighn (sold by the company Invitrogen), containing 10% by weight of fetal calf serum and 2.5 ml of a solution of antibiotics (penicillin/streptomycin) at 50 IU/g. The OCDO tested comes from the company Steraloids. In order to study in vitro the effect of OCDO on cell growth, the TT cells are seeded on to six-well plates (200 000 cells per well) in an F12K medium as defined above. The TT cells are treated every two days either with a solvent (ethanol at 0.1% in PBS phosphate buffer) or with amounts of OCDO in the solvent resulting in concentrations of 0.1, 1, 2.5 or 5 μM. The cells are counted 4 days after seeding. As is seen in FIG. 7, the proliferation of the TT cells is increased in a concentration-dependent manner by the treatment with OCDO, when compared, as reference, with the proliferation of the cells treated with the solvent. At the OCDO concentration of 5 μM, a proliferation induction factor of 1.5 is measured after 4 days of treatment; the induction of proliferation is therefore established in vitro for OCDO concentrations of between 100 nM and 5 μM. The proliferation-stimulating factor is comparable to that of estrogens, which are molecules that have mitogenic properties on mammary tumor cells (see: Medina et al., J Pharmacol Exp Ther, vol. 319, 2006: p. 139-149).
b) The applicants also studied in vivo the effect of OCDO on tumor development. For injection into animals, the TT cells defined above in this example under a) are recovered with trypsin, washed twice and suspended in a PBS phosphate buffer. The TT cells (approximately 4×10⁶ cells/0.1 ml) are then injected subcutaneously into the flank of 6-week-old “Swiss nude nu/nu” female mice (supplied by Charles River). The animals are treated subcutaneously once a day, on 5 days out of 7, either with OCDO at the dose of 50 μg/kg (treated group) or with the solvent (control group) over a period of 110 days (the solvent used is 0.1% ethanol in phosphate buffer (PBS)). The animals (5 or 10 mice per group) are monitored regularly in order to measure tumor development. The volume of the tumors is calculated according to the formula L×l²×0.5, where L is the length and l is the width of the tumor. FIG. 8 represents the volume V of the tumors as a function of treatment time t. The treatment with OCDO significantly accelerates the tumor growth; the tumor volume in the animals treated with OCDO is almost three times larger than for the control animals treated with the solvent.

The animals were euthanized after 75 days of treatment and the tumor and the various organs were removed in order to be analyzed histologically. Twice as much invasion of the lymph nodes (LN) was observed in the animals treated with OCDO compared with the animals treated with the solvent (see FIG. 9).

A histomorphological analysis was performed. To do this, the tumors of the mice treated with OCDO or with the solvent and also various organs (lymph nodes, lung and liver) are removed, fixed in 10% buffered neutral formalin and embedded in paraffin blocks. For these analyses, the sections are stained with hematoxylin and eosin. The immunolabeling is carried out with antibodies directed against various human antigens associated with medullary thyroid carcinomas. The antibodies used are the anti-calcitonin polyclonal antibody (Dako SAS, Trappes, France, 1:1000), the anti-cytokeratin monoclonal antibody (Dako clone AE1/AE3, 1:50) and the anti-epithelial membrane antigen (EMA) monoclonal antibody (Dako clone E29, 1:50). The immunolabeling of the paraffin sections is preceded by an antigen recovery technique by heating in a citrate buffer (10 mM, pH 6) either twice for 10 minutes in a microwave oven (750 W) for the anti-CEA antibody, or in a waterbath at 95° C. for 40 minutes for the anti-calcitonin and anti-EMA antibodies.

After incubation with the antibodies defined above, the sections are immunolabeled with the streptavidin-biotin-peroxidase complex (StreptABComplex/HRP, Dako) followed by a chromogenic solution of diaminobenzidine (DAB), and are then stained with hematoxylin.

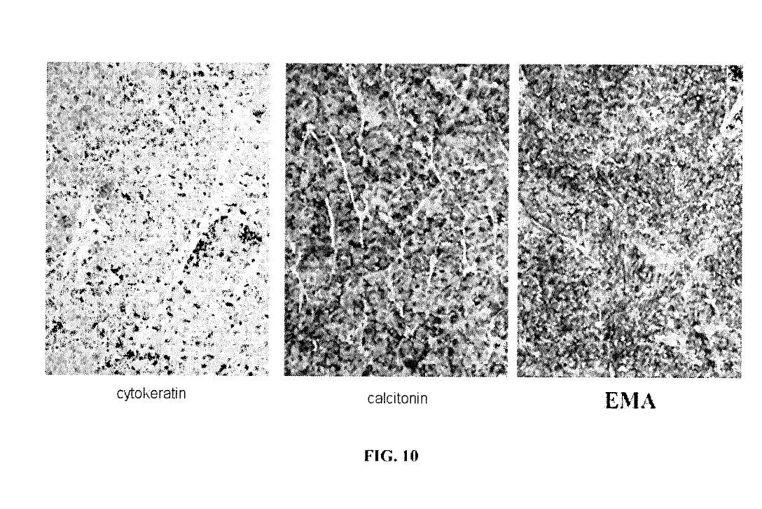

The negative controls are performed by incubation in a buffer solution not containing the primary antibody. The results obtained by means of this histological analysis are represented in FIG. 10 for each of the three antibodies used. It is noted that the tumors derived from the animals treated with OCDO are indeed human medullary thyroid carcinomas, which confirms the invasion of the lymph nodes by cells derived from the tumor.

The applicants therefore established that OCDO has a mitogenic activity in vivo by stimulating implanted tumor growth in laboratory animals.

### EXAMPLE 3: OCDO STIMULATES IL10 AND REDUCES IL12, WHICH EXPLAINS ITS CARCINOGENIC ACTION

The applicants, having established, according to the invention, that OCDO stimulates the invasive capacity of cancer cells in vivo, confirmed this effect of OCDO by studying, in vitro, the expression of cytokines on THP1 cells treated in vitro with OCDO.

The THP1 cells (human myeloid cell line supplied by ATCC) are cultured with a culture medium of DMEM type (Dulbecco's modified eagle medium) supplemented with 10% of fetal calf serum and a mixture of antibiotics. The THP1 cells are treated with 10 μM of OCDO for 4 hours. The RNAs are extracted according to a conventional procedure. The complementary DNAs are produced, and then used to measure the expression of the genes of interest. The expression of the genes encoding interleukin 12 (IL12) and interleukin 10 (IL10) were studied, these interleukins representing a pair of cytokines with antagonistic properties. IL12 is an immunostimulatory cytokine, whereas IL10 is an immunosuppressive cytokine. The expression ratio of these two cytokines makes it possible to evaluate the immunosuppressor or immunostimulatory potential of the cell and its ability to promote tumor progression or, on the contrary, to slow it down.

The primers used correspond to the human sequences of IL10 and of IL12 and are the following:

IL10 sense:
(SEQ ID NO: 1)
AAA-CCA-AAC-CAC-AAG-ACA-GAC,
IL10 anti-sense:
(SEQ ID NO:2)
GCT-GAA-GGC-ATC-TCG-GAG,
IL12 sense:
(SEQ ID NO: 3)
CTA-TGG-TGA-GCC-GTG-ATT-GTG,
IL12 anti-sense:
(SEQ ID NO: 4)
TCT-GTG-TCA-TCC-TCC-TGT-GTC.

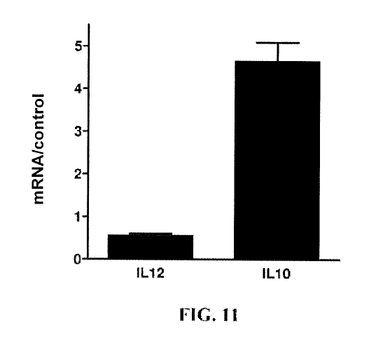

The results are represented in FIG. 11: OCDO stimulates the expression of the immunosuppressive cytokine IL10 and reduces the expression of the cytokine IL12. This mechanism makes it possible to explain the stimulation by OCDO of the invasive capacity of tumor cells noted via example 2.

### EXAMPLE 4: ASSAYING OF OCDO

### A) Assaying by HPLC

The OCDO was separated and assayed by a high performance liquid chromatography HPLC method (95 MeOH/5 H₂O; 0.7 ml/min; column Ultrasep ES 6 μm of 250×4, C18 (Bishoff, Leonberg, Germany)). The chromatograph is an apparatus from the company Perkin Elmer; it comprises a series 200 pump and a diode array detector of type 200.

The apparatus is equipped with a “PC” computer, which uses the Turbochrome™) for apparatus control and data processing.

A cell extract of cultured MCF7 tumor cells is prepared as indicated in assay 1: approximately 60 million cells are lyzed so as to obtain 25 μl of liquid extract after centrifugation for 10 min at 1200 rpm. 20 μl of this extract are passed through the column of the chromatograph.

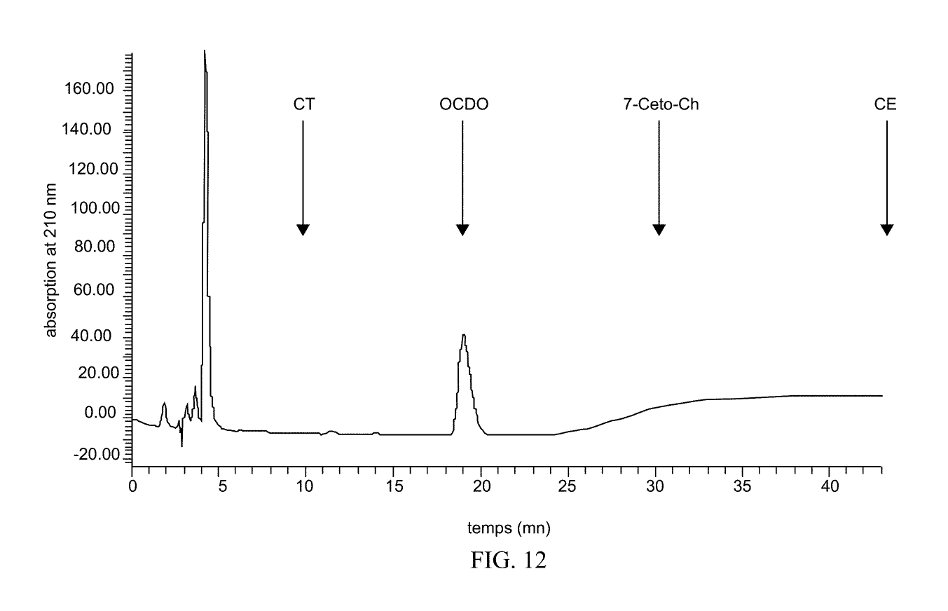

The resulting chromatogram is provided in FIG. 12: it is seen that the OCDO is separated from the CT, from the CE and from the keto-cholesterol (7-keto-Ch). The retention time of the OCDO is 19 min.

A calibration is performed in order to link the measurement of the surface area of the OCDO peak thus obtained and the weight of the OCDO contained in the sample.

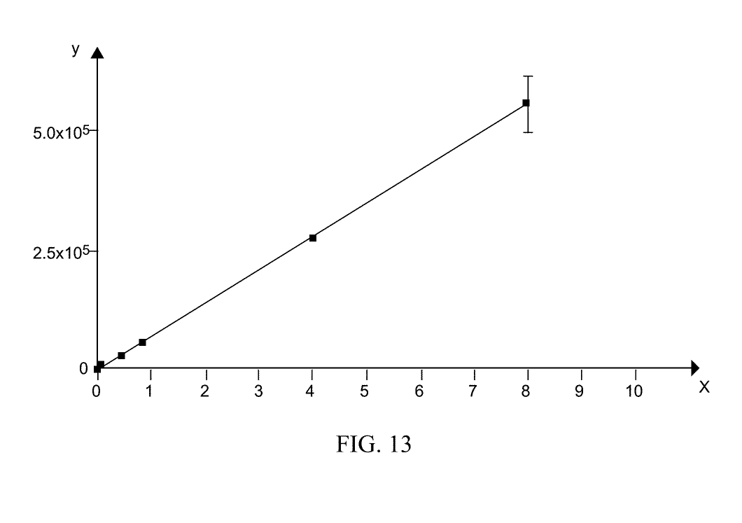

This calibration is carried out using ethanolic solutions of OCDO sold by the company Steraloids, of increasing concentrations. A fixed volume of 20 μl of sample is used. The following amounts of OCDO were injected: 80 ng, 0.4 μg, 0.8 μg, 4 μg and 8 μg. The area of the peaks corresponding to the OCDO was measured by integration using the Turbochrome™ software; these values (y) were reported on the graph of FIG. 13 as a function of the corresponding OCDO masses (x). A suitable correlation straight line is obtained (y=69430x+1381; r²=0.999; n=6×3; p<0.0001; x in μg of OCDO; y in μV·s) making it possible to quantify the OCDO for masses of between 40 ng and 80 μg. This method makes it possible to quantify the OCDO for amounts of between 0.1 and 5 μg of molecules.

B) Assaying by Gas Chromatography Coupled with Mass Spectrometry (GC/MS)

A mixture of 5α-cholestane and OCDO is trimethylsilylated according to the method described by Kedjouar (see Kedjouar et al., J Biol Chem, 2004, vol. 279, No. 32, p. 34048-61). The sample is treated with 20 a mixture of N,O-bis(trimethylsilyl)-trifluoroacetamide/pyridine (50:50, v/v) for 30 min at a temperature of 60° C. The reagents are evaporated under a stream of nitrogen and the trimethylsilylated (TMS) derivatives are dissolved in hexane. These GC/MS analyses are carried out on a Hewlett Packard system apparatus type 4890 equipped with an RTX-50 silica capillary column (30 m×0.32 mm internal diameter, film of thickness 0.1 μm; Restek). The oven temperature was programmed at 230° C. for 1 minute, then from 230 to 240° C. at a rate of increase of 1° C. per minute for 10 minutes, from 240° C. to 250° C. at a rate of increase of 3° C. per minute, then up to 290° C. at a rate of increase of 45° C./min then to 330° C. in 1 minute.

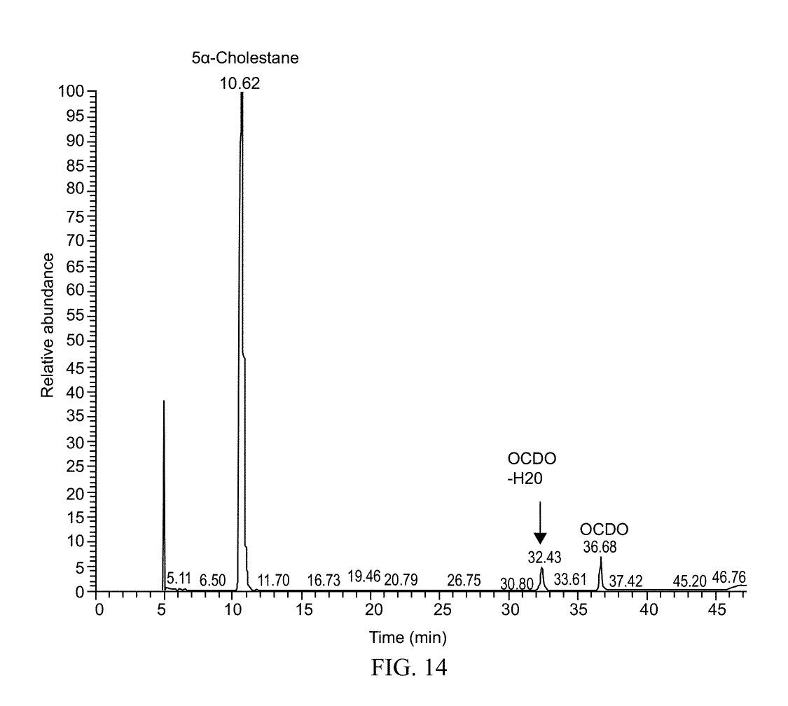

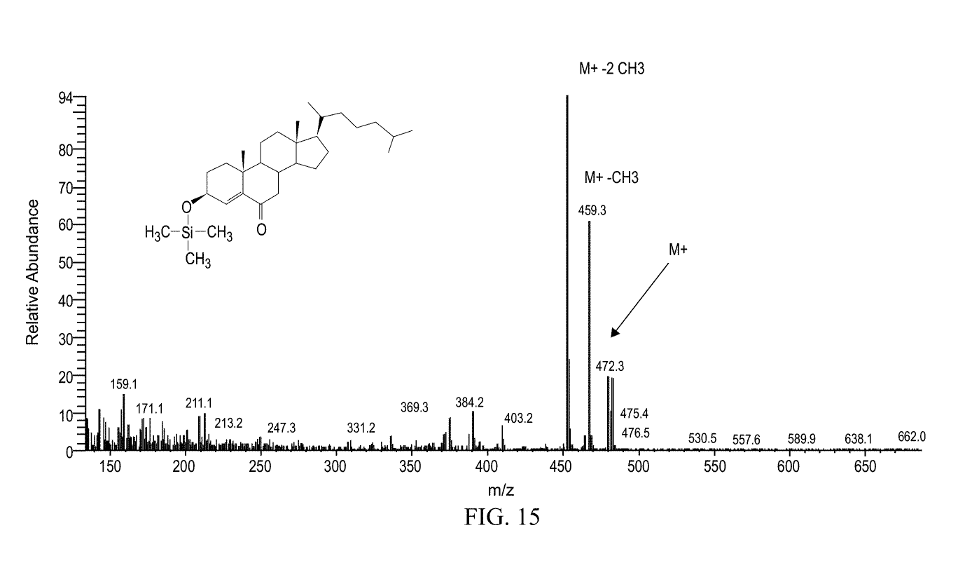

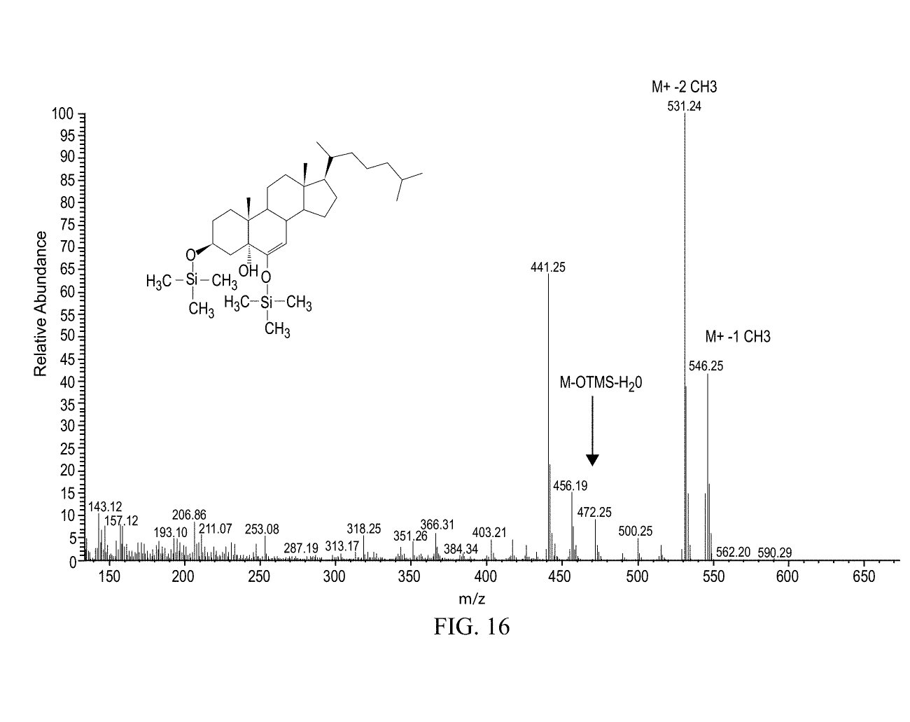

The injector and the detector were at 310° C. and 340° C., respectively. The GC profile obtained is represented in FIG. 14. Three peaks were obtained. The first corresponds to 5α-cholestane, which has a retention time of 10.62 minutes. The second corresponds to a retention time of 32.43 minutes and gives, in analysis by mass spectrometry with electron impact fragmentation, a result which corresponds to an OCDO dehydration product (M+(472.3)), M+ minus a CH₃ group and M+ minus 2 CH₃ groups (see FIG. 15). Finally, the last peak (retention time of 36.68 minutes) corresponds to the OCDO and its mass fragmentation profile is given in FIG. 16. This profile determines the masses of 546.2 (M+ minus a CH₃), 531.2 (M+ minus two CH₃) and 472.2 (M+ minus H₂O and minus an OTMS group).

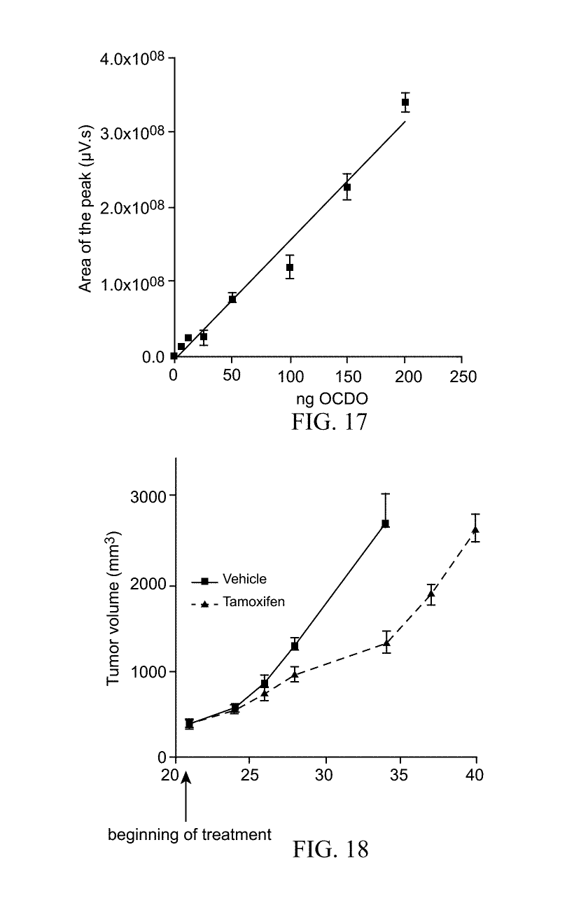

The calibration of the method is carried out using ethanolic solutions of OCDO of increasing concentrations. The following amounts of OCDO were injected: 6.25 ng, 12.5 ng, 50 ng and 100 ng. Integration of the area of the peaks corresponding to the OCDO for these various amounts of OCDO made it possible to establish a calibration curve for quantifying the OCDO. This method makes it possible to assay amounts of OCDO of between 5 and 200 ng (see FIG. 17).

### EXAMPLE 5: INHIBITION OF OCDO PRODUCTION BY TAMOXIFEN IN VIVO

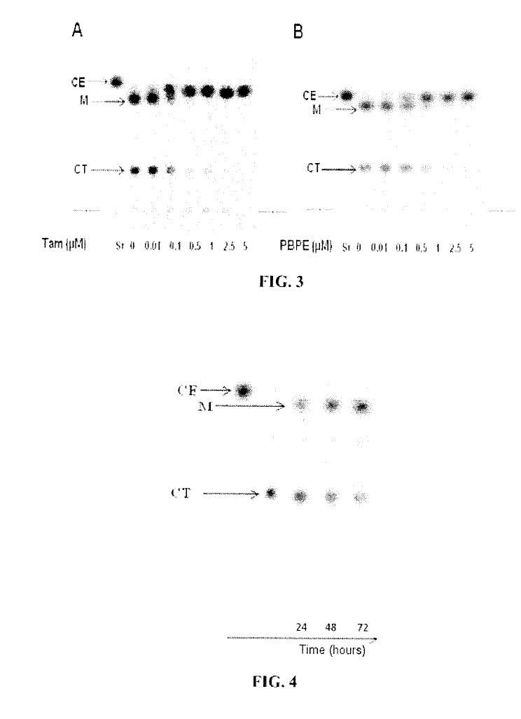

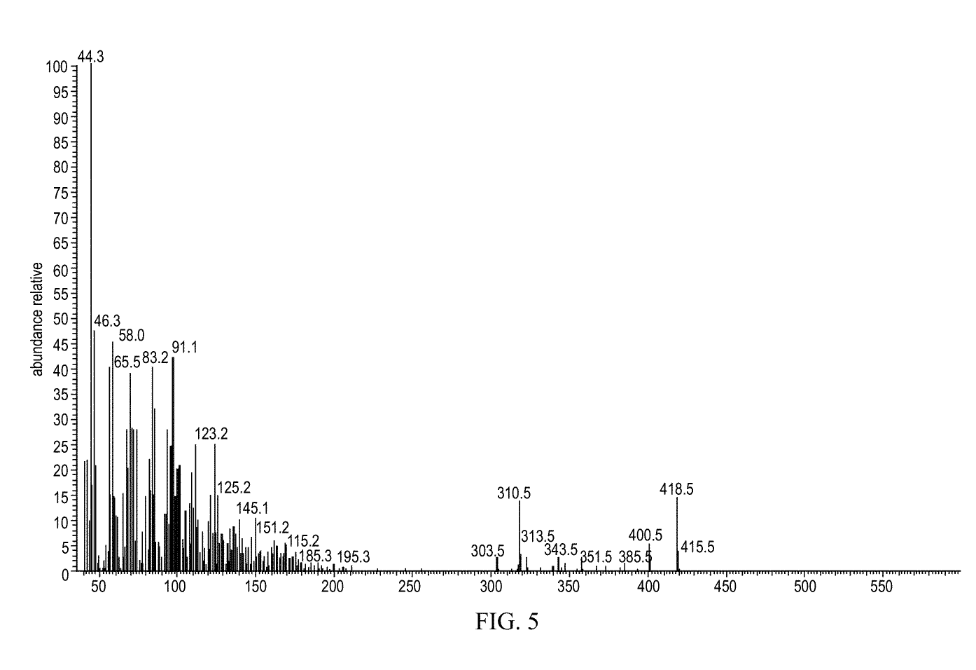

As has already been shown in assay 2 (FIG. 3A) that the addition of tamoxifen to MCF7 tumor cell cultures blocks OCDO production in these cells. It will be shown hereinafter that tamoxifen administered in vivo also causes an inhibition of OCDO production.

The assaying techniques used in example 4 were used for measuring the modulation of OCDO on tumors implanted in mice.

A cell culture of TS/A cells was first performed. These TS/A cells are mouse mammary adenocarcinomas (see Nanni P et al., Clin. Exp. Metastasis, 1983 October-December; 1(4): 373-80). The cells are cultured, at 37° C. under 5% CO₂, in a DMEM medium supplemented with 2 g/liter of sodium carbonate, 1.2 mM of glutamine (pH 7.4 at 23° C.), 5% of fetal bovine serum (Gibco) and 2.5 ml of antibiotics per liter of medium (penicillin/streptomycin).

The TS/A cells are recovered with trypsin, washed twice and suspended in PBS buffer. The TS/A cells (approximately 4×10⁶ 15 cells/0.1 ml) are then injected subcutaneously into the flank of 6-week-old female BALB/c mice (supplied by Charles River). The animals are treated for 27 days after the implantation of the tumors, intratumorally once a day over a period of 37 days, either with tamoxifen at a concentration of 10 μM for injection volumes of 100 μl (group treated with tamoxifen), or with the vehicle solvent (ethanol at 0.1% in PBS phosphate buffer) (control group). The animals (10 mice per group) are monitored regularly for tumor development. The volume of the tumors is calculated according to the following formula: L×l²×0.5 (L is the length and l is the width of the tumor). The experiment was reproduced twice. The results are given in FIG. 18 (the arrow indicates the beginning of the treatment).

An inhibition of tumor growth in the mice treated with tamoxifen was thus qualitatively observed in FIG. 18.

In order to obtain quantitative information on the inhibition of OCDO, the experiment was reproduced under the same conditions and the tumors were removed on day 28. 5 volumes (relative to the mass removed) of buffer (50 mM Tris-HCl, 150 mM KCl, pH=7.4) are added. The tumors are ground and the homogenates are centrifuged for 5 minutes at 2500 rpm at 4° C. The supernatants are recovered and then 1 volume of methanol (relative to the volume of supernatant) and 2 volumes of chloroform are added. After centrifugation (to separate the phases), the organic phase is recovered. The organic phase is evaporated to dryness and then the residue is taken up with 0.5 ml of chloroform. The organic extracts are filtered on 0.5 ml silica cartridges. The polar sterols are eluted sequentially:

1) 0.5 ml of a hexane/chloroform mixture (1/1),
2) 0.5 ml of chloroform,
3) 0.5 ml of ethyl acetate and methanol.

The addition of a ¹⁴C-labeled OCDO external standard during the extraction and purification phases makes it possible to establish a recovery yield of 86±6%. The ethyl acetate fractions are evaporated, silylated (50 μl 1/1 acetonitrile/BSTFA) and then analyzed by GC/MS (2 μl) according to the conditions described above.

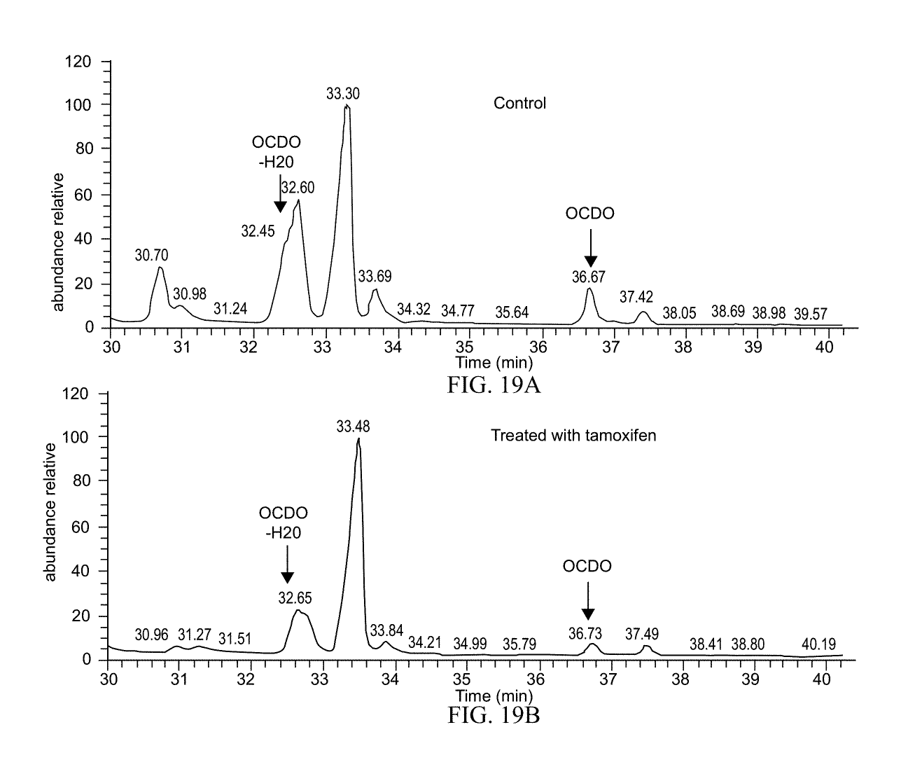

FIG. 19 shows two chromatograms prepared in the gas phase as indicated in example 4, corresponding:

A) to an extract of tumor originating from a control animal: the tumor had a mass of 2 g;
B) to an extract of tumor originating from an animal treated with tamoxifen: the tumor had a mass of 1.45 g.

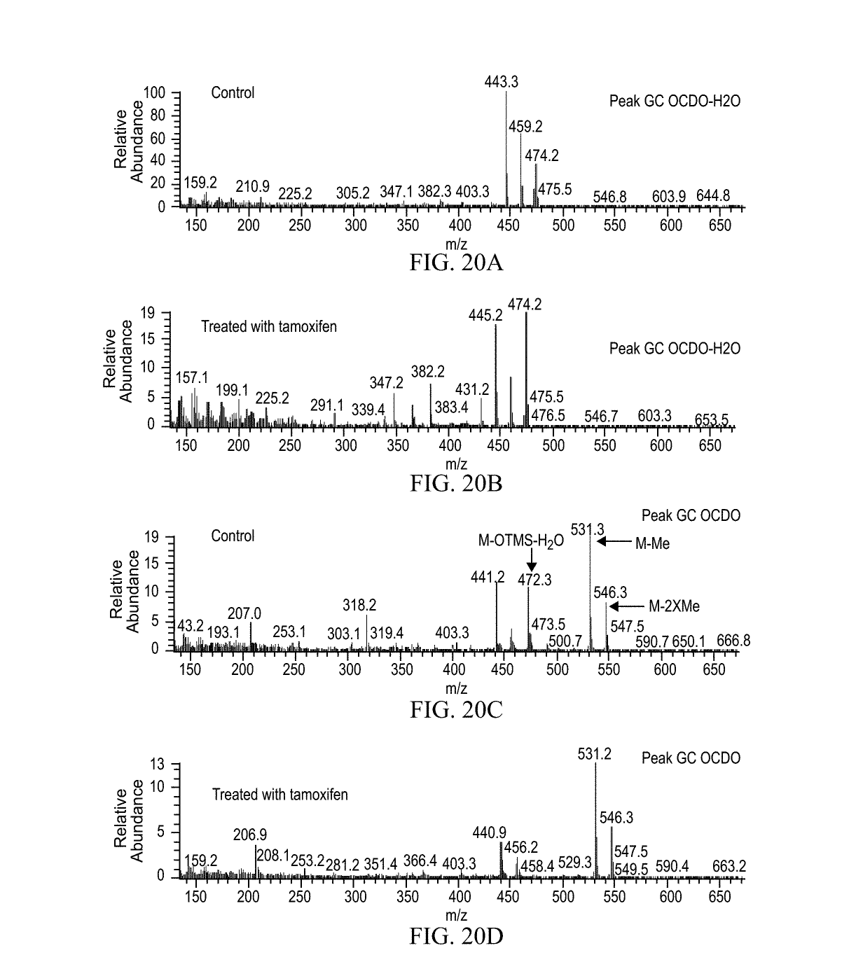

A significant decrease in the peaks originating from the OCDO in the tumor of the treated mice (B) compared with that of the nontreated mice (A) is observed in FIG. 19. The mass spectrometry analysis confirms the structure of the molecules in the peaks of interest (see FIG. 20).

The results thus obtained, in terms of numbers, have been given below, the control group having been previously defined, and the values associated with the control have been valued at 100 with coefficients which were also applied to the results relating to tamoxifen.

| | Day 28 | | Day 37 | |
|---|---|---|---|---|
| | Tumor size | OCDO | Tumor size | OCDO |
| | (% | (% | (% | (% |
| Molecule | control) | control) | control) | control) |
| Nontreated | 100 | 100 | 100 | 100 |
| control | | | | |
| Tamoxifen | 73.5 ± 8 | 19.2 ± 4 | 46.2 ± 7 | 8.4 ± 4 |

It is seen that, in vivo, tamoxifen significantly inhibits OCDO.

The fact that tamoxifen exerts an antitumor activity while at the same time inhibiting, in vivo, the production of a tumor-promoting oxysterol shows that OCDO plays a role in the effects of tamoxifen.

### EXAMPLE 6: INHIBITION OF OCDO PRODUCTION BY PBPE IN VIVO

PBPE is a compound of which the antiproliferative effect was established by in vitro experiments (see the reference cited in assay 2 and also the publication Payre B. et al., Mol. Cancer Ther., 7(12), 3707-3717). In vivo results analogous to those present in example 5 for tamoxifen were determined for PBPE. The protocol used is strictly identical to that which was detailed in example 5 for the treatment with tamoxifen, with the only difference that the daily intratumor injections are carried out at a concentration of 40 μM for PBPE for injection volumes of 100 μl. The results are collated in table 5 below:

| | Day 28 | | Day 37 | |
|---|---|---|---|---|
| | Tumor size | OCDO | Tumor size | OCDO |
| | (% | (% | (% | (% |
| Molecule | control) | control) | control) | control) |
| Nontreated | 100 | 100 | 100 | 100 |
| control | | | | |
| PBPE | 71.6 ± 7 | 22.5 ± 5 | 54.5 ± 8 | 9.6 ± 5 |

It was thus determined in vivo that PBPE both inhibits OCDO and causes tumors to regress.

### EXAMPLE 7: INHIBITION OF OCDO PRODUCTION BY PBPE ON VARIOUS TUMOR CELLS

Assay 2 provided above showed that cell extracts of MCF7 tumor cells incubated for 3 days in PBPE solutions lead to the observation of an inhibition of the metabolite that was identified as being OCDO.

An analogous experiment was reproduced with different cell lines, with the cells tested being incubated for 48 hours in a 40 μM solution of PBPE. It was noted that the OCDO was 100% inhibited for all the lines in the following table that were tested:

| | | Inhibition of |
|---|---|---|
| Cell line | Type | OCDO production |
| MCF-7 | Human mammary carcinoma | 100% |
| MDA-MB-231 | Human mammary carcinoma | 100% |
| A-549 | Human lung carcinoma | 100% |
| B-16-F10 | Murine melanoma | 100% |
| U-937 | Human leukemia | 100% |
| HT-29 | Human colon carcinoma | 100% |
| HeLa | Human uterine carcinoma | 100% |
| C6 | Rat glyoma | 100% |
| SH-N-SH | Human neuroblastoma | 100% |
| Saos-2 | Human osteosarcoma | 100% |
| P19 | Murine carcinoembryonic | 100% |
| | line | |
| TT | Human medullary thyroid | 100% |
| | carcinoma | |

Generally, in order to establish the percentages of OCDO inhibition from the thin-layer chromatography (TLC) plates obtained as in assay 2, the radioactive metabolites are identified and quantified on the basis of said plates using a europium-sensitive plate of GP Phosphor screen type (GE Healthcare) and a Storm 840 phosphorimagor (GE Healthcare). The proportion of radiolabeled oxysterols is determined on the autoradiogram obtained by densitometry using the Image Quant 5.2 software. The percentage is calculated on the basis of the ratio between the amount of oxysterols quantified divided by the sum of the amounts of oxysterols (CEE+CE+CT+OCDO). Since the CEE (CE ester) originates exclusively from the CE, the percentage CE is calculated on the basis of the ratio (CE+CEE)/(CE+CEE+CT+OCDO).

### EXAMPLE 8: OCDO IS INHIBITED WHEN CHEH IS INACTIVE

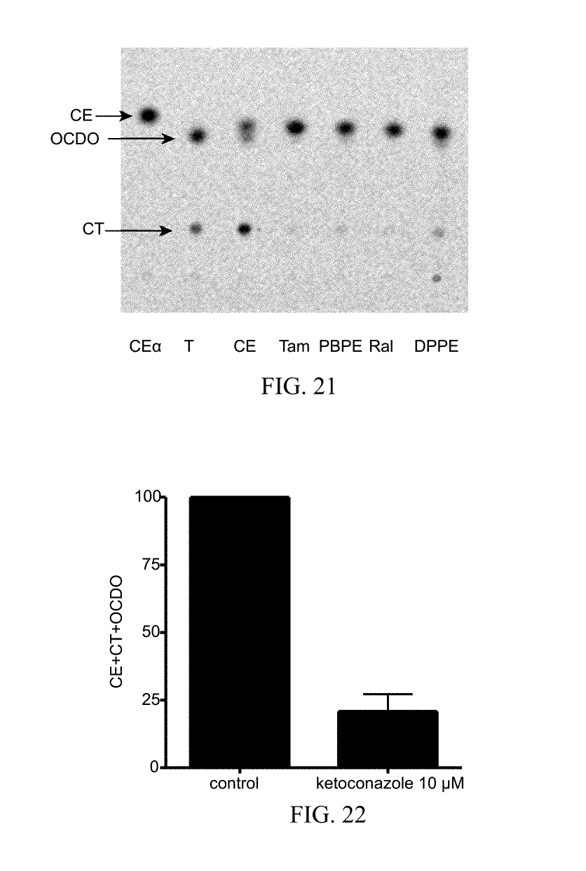

As was previously established in this patent application, it is known that tumor cells produce OCDO, which implies that the ChEH hydrolase is active since it enables the conversion of CE to CT, which is the obligatory change for the production of OCDO. As will be established in table 1 given later in this example, tamoxifen, PBPE, raloxifene and DPPE are ChEH inhibitors. Assay 2 given above was therefore completed by measuring the amounts of the products present when the MCF7 tumor cells are incubated as indicated in detail in assay 2. The calculation of the percentages of CE, CT and OCDO was carried out as indicated in example 7. The results are given in FIG. 21, which shows a thin-layer chromatography plate on which the lanes correspond to incubations of the cells with the vehicle solvent T (0.1% ethanol in PBS buffer), with CE- and with four ChEH inhibitors, the identifiers of which are given at the bottom of the lanes. The numerical values obtained are collated in the table below:

| Compounds | % CE | % OCDO | % CT |
|---|---|---|---|
| EtOH (T) | 0 | 73.1 | 26.9 |
| CEα 10 μM | 31.5 | 27 | 41.5 |
| Tam 2.5 μM | 90.3 | 6.9 | 2.8 |
| PBPE 10 μM | 89.2 | 6.2 | 4.6 |
| Ral 10 μM | 96.5 | 0.5 | 3 |
| DPPE 1.5 μM | 80.7 | 8.8 | 10.5 |

T = control
DPPE = N,N-diethyl-2-(4-benzylphenoxy)ethanamine
Ral = raloxifene

It is noted that inhibition of ChEH indeed leads to inhibition of OCDO, the CT product being very reduced in amount.

In fact, the formation of OCDO in a cell depends on several parameters:
1. the presence of cholesterol,
2. the presence of CE,
3. the presence of cholesterol epoxide hydrolase (ChEH),
4. the presence of CT,
5. the transport of CT to the zone where CT is oxidized to OCDO,
6. the presence and the activity of the enzyme responsible for the oxidation of CT to OCDO.

In order to establish that the link that exists between, on the one hand, the ability of various compounds to inhibit OCDO in an MCF7 tumor cell and, on the other hand, their quality as a ChEH inhibitor, is well-founded, the inhibition coefficients Ki for ChEH relating to a certain number of products was calculated and the production of OCDO by MCF7 cells after incubation of the cells with the same products was measured according to the protocol defined in assay 2 of the present patent application. All of the results are given in tables 1 and 2. Based on these two tables, it appears that any inhibitor of an enzyme involved in cholesterol biosynthesis causes a decrease in the cholesterol that can be used for OCDO production. Inhibitors of enzymes from hydromethylglutaryl coenzyme A synthetase (HMG CoA synthetase) to 7-dehydrocholesterol reductase and 24-dehydrocholesterol reductase will in particular be noted.

An experiment was carried out among this category of inhibitors, namely with an HMG-CoA reductase inhibitor, such as lovastatin, used at a concentration of 10 μM: it was noted that, after incubation of MCF7 cells according to the protocol given in assay 2 and by bringing the control to the value 100, an OCDO production by the MCF7 cells of less than 1 is obtained.

TABLE 1

| | Molecule | Kᵢ ChEH (nM) |
|---|---|---|
| AEBS ligands | PBPE | 26.8 ± 6 |
| | PCPE | 34.7 ± 8 |
| | Tesmilifene | 62.4 ± 3 |
| | DDA | 1250.4 ± 6 |
| Estrogen receptor | Tamoxifen | 33.6 ± 8 |
| modulators | 4OH-Tamoxifen | 145.3 ± 4 |
| | Raloxifene | 35.6 ± 4 |
| | Nitromiphene | 17.7 ± 6 |
| | Clomiphene | 9.0 ± 2 |
| | RU 39,411 | 155.2 ± 8 |
| Sigma receptor | BD-1008 | 98.7 ± 9 |
| ligands | Haloperidol | 18067 ± 14 |
| | SR-31747A | 6.2 ± 2 |
| | Ibogaine | 2150 ± 11 |
| | AC-915 | 3527 ± 9 |
| | Rimcazole | 2325 ± 8 |
| | Amiodarone | 733.1 ± 9 |
| | Trifluoroperazine | 135.4 ± 7 |
| Cholesterol | Ro 48-8071 | 88.9 ± 5 |
| biosynthesis | U-18666A | 90.3 ± 5 |
| inhibitors | AY-9944 | 649 ± 6 |
| | Triparanol | 39.5 ± 3 |
| | Terbinafine | 9105 ± 33 |
| | SKF-525A | 1904 ± 11 |

TABLE 2

| | | OCDO |
|---|---|---|
| | | production by |
| | Molecule | MCF-7 |
| | (concentration in μM) | (% control) |
| Control | — | 100 |
| AEBS ligands | PBPE (1) | <1 |
| | PCPE (1) | <1 |
| | Tesmilifene (1) | <1 |
| | DDA | <1 |
| Estrogen receptor | Tamoxifen (1) | <1 |
| modulators | 4OH-Tamoxifen (1) | <1 |
| | Raloxifene (1) | <1 |
| | Nitromiphene (1) | <1 |
| | Clomiphene (1) | <1 |
| | RU 39,411 (5) | <1 |
| σ receptor | BD-1008 (1) | <1 |
| ligands | Haloperidol (100) | 42 |
| | SR-31747A (1) | <1 |
| | Ibogaine (5) | 25 |
| | AC-915 (10) | 16 |
| | Rimcazole (5) | 12 |
| | Amiodarone (5) | 8 |
| | Trifluoroperazine (1) | <1 |
| Cholesterol | Ro 48-8071 (10) | <1 |
| biosynthesis | U-18666A (1) | <1 |
| inhibitors | AY-9944 (5) | <1 |
| | Triparanol (1) | <1 |
| | Terbinafine (10) | <1 |
| | SKF-525A (10) | <1 |

The products which appear in tables 1 and 2 and which have not been previously identified in the present text are defined by their chemical name in the following list:
PCPE: 1-(2-(4-(2-phenylpropan-2-yl)phenoxy)ethyl)-pyrrolidine;
tesmilifene: 2-(4-benzylphenoxy)-N,N-diethylethanamine; tamoxifen: 2-[4-[(Z)-1,2-di(phenyl)but-1-enyl]phenoxy]-N,N-dimethylethanamine;
4OH-tamoxifen: 4-[(Z)-1-[4-(2-dimethylaminoethyloxy)-phenyl]-2-phenylbut-1-enyl]phenol;
raloxifene: [6-hydroxy-2-(4-hydroxyphenyl)-1-benzothiophen-3-yl]-[4-(2-piperidin-1-ylethoxy)phenyl]methanone;
nitromiphene: 1-[2-[4-[(Z)-1-(4-methoxyphenyl)-2-nitro-2-phenylethenyl]phenoxy]ethyl]pyrrolidine;
clomiphene: 2-[4-[(Z)-2-chloro-1,2-di(phenyl)ethenyl]-phenoxy]-N,N-diethylethanamine;
RU 39411: 11-[4-N,N-[diethylaminoethoxy]phenyl]estra-1,3,5(10)triene-3,17-diol.
BD-1008: N-(3,4-dichlorophenethyl)-N-methyl-2-(pyrrolidon-1-yl)ethanamine;
haloperidol: 4-(4-(4-chlorophenyl)-4-hydroxypiperidin-1-yl)-1-(4-fluorophenyl)butan-1-one; SR-31747A: (E)-N-(4-(3-chloro-4-cyclohexylphenyl)but-3-enyl)-N-ethylcyclohexanamine;
AC915: N-(3,4-dichlorophenethyl)-N-methyl-2-(pyrrolidon-1-yl)ethanamine;
rimcazole: 9-[3-[(3S,5R)-3,5-dimethylpiperazin-1-yl]propyl]carbazole;
amiodarone: (2-butyl-1-benzofuran-3-yl)-[4-(2-diethylaminoethyloxy)-3,5-diiodophenyl]methanone;
trifluoroperazine: 10-[3-(4-methylpiperazin-1-yl)propyl]-2-(trifluoromethyl)phenothiazine;
RO 48-8071: (4-(6-(allyl(methyl)amino)hexyloxy)phenyl) (4-bromophenyl)-methanone;
U-18666A: 3-beta-(2-(diethylamino)ethoxy)androst-5-en-17-one;
AY-9944: trans-1,4-bis(2-chlorobenzaminomethyl)-cyclohexane;
triparanol: 2-(4-chlorophenyl)-1-(4-(2-diethylamino)-ethoxy)phenyl)-1-p-tolylethanol;
terbinafine: (E)-N,3,6,6-tetramethyl-N-(naphthalen-1-ylmethyl)hept-2-en-4-yn-1-amine;
SKF-525A: 2-diethylaminoethyl-2,2-diphenylpentanoate.

The inhibitors which were the subject of experiments having results in tables 1 and 2 include estrogen receptor modulators, anti-estrogen membrane binding site (AEBS) ligands, sigma-1 and -2 receptor ligands, and inhibitors of cholesterol biosynthesis from squalene synthetase up to 7- and 24-dehydrocholesterol reductase. All these inhibitors have one thing in common, namely that of being AEBS ligands.

The protocols used for measuring the inhibition coefficients Ki of table 1 are given in detail hereinafter:

a) Ki for the AEBSs

The Ki is the inhibition constant corresponding to the molecule of interest and is measured in the following way: rat liver microsomes are incubated with a concentrations of 2.5 nM of tritiated tamoxifen (supplied by the company GE Healthcare) and increasing concentration of inhibitor of between 0.01 and 1000 μM under the conditions described in the following publication: Poirot M. et al., Bioorg Med Chem 2000, vol. 8(8), p. 2007-2016. The IC50 values correspond to the concentration of inhibitor required to inhibit 50% of the activity of the ChEH; they are determined using a GraphPad Prism (version 4) data processing program. The Ki values are calculated using the Cheng-Prussof equation (Cheng and Prussof, Biochem Pharmacol, 1973, vol. 22(23), pages 3099-3108). The Ki is expressed according to the equation: Ki=[IC50](1+(tritiated tamoxifen])/Kd). The concentration of tritiated tamoxifen is 2.5 nM and the dissociation constant at equilibrium of the tritiated tamoxifen for AEBS is 2 nM.

b) Determination of the Ki for ChEH:

150 μg of rat liver microsome proteins are incubated in the presence of 2 concentrations of [¹⁴C]CEα with increasing concentrations of inhibitors of between 0.01 and 1000 μM under the conditions described above for measuring the ChEH activity. The Ki is measured as the projection on the x-axis of the intersection of the lines obtained by reporting on a graph the values of 1/V as a function of 1/S for ChEH, as determined by the Dixon method (Dixon M, Biocheml Jl, 1953, vol. 55(1), p. 170-171).

### EXAMPLE 9: INHIBITION OF OCDO BY A CYTOCHROME P450 INHIBITOR

Cholesterol epoxidation can be produced by self-oxidation of cholesterol with oxygen in the air, under the action of enzymes such as cytochromes P450 or lipoxygenases (see Schroepfer G. Jr, Physiological Reviews, vol. 80, No. 1, p. 361-554, 2000).

An inhibition of the production of the epoxysterol CE and of its derivatives, which are CT and OCDO, was noted when using a general cytochrome P450 inhibitor, namely ketoconazole (see FIG. 22).

The protocol for this experiment is the same as that described in example 7.

### EXAMPLE 10: INHIBITION OF CHEH BY AN AMINOALKYL STEROL

For this experiment, an aminoalkyl sterol included in French patent 2 838 741 (and also see: de Medina et al., J Med Chem: 2009, vol. 52, No. 23, p. 7765-7777), having the formula: 6-N-[2-(3H-imidazol-4-yl)ethylamino]cholestane-3β,5α-diol (DDA), was chosen. MCF7 tumor cells were incubated with [¹⁴C]CE (10 mCurie/mmol, 0.6 μM) for 48 hours in the presence or absence of the abovementioned aminoalkyl sterol (at the concentrations of 0.1 and 1 μM).

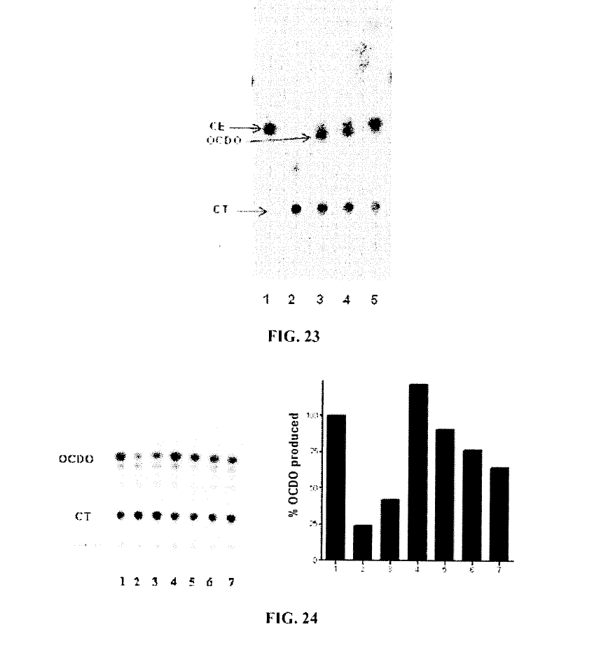

A thin-layer chromatography was carried out, the plate of which has been reproduced in FIG. 23. On this plate, the incubation is carried out, for lane 1, with [¹⁴C]CE; for lane 2, with [¹⁴C]CT; for lane 3, with the vehicle solvent, which is the same as that used for assays 1 to 5; for lane 4, with [14C]CE and 0.1 μM of the aminoalkyl sterol; and for lane 5, with [¹⁴C]CE and a concentration of 1 μM of the aminoalkyl sterol.

It is observed that the presence of the aminoalkyl sterol causes an inhibition of ChEH; this inhibition is dependent on the concentration of aminoalkyl sterol.

Moreover, a study of this inhibition on homogenate was also carried out. The protocol is as follows: the MCF7 cells are detached with trypsin and taken up with RPMI medium containing 5% FBS. The cell suspension obtained (60 million cells) is centrifuged, washed with cold PBS and resuspended in 1 ml of 20 Tris-HCl buffer (pH=7.4; 150 mM KCl). The cells are lyzed by means of five freezing/thawing (liquid nitrogen/37° C.) cycles. The solution is centrifuged at 1200 rpm at 4° C. for 10 minutes. The supernatant is recovered and the amount of proteins is determined by the Bradford method. The measurement of the ChEH activity on MCF7 cell lysate is carried out as follows: the enzymatic activity is measured on 150 μg of proteins in a final volume of 150 μl containing 125 μl of ChEH buffer (Tris-HCl, pH 7.4, 150 mM KCl) and 15 μl of MCF7 proteins. The IC50 values were compared and it was noted that (IC50)_{cells}=0.6 μM, whereas (IC50)_{homogenate}=11.2 μM.

This difference between the IC50 values shows that the aminoalkyl sterol tested exhibits properties of preventing the occurrence of cancers.

In addition, for the DDA compound, results analogous to those present in example 6 for PBPE were determined. The protocol used is strictly identical to that which was described in detail in example 5 for the treatment with tamoxifen, with the one difference that the daily intratumor injections are carried out at a concentration of 10 μM for injection volumes of 100 μl. The results are collated in the table below:

| | Day 28 | | Day 37 | |
|---|---|---|---|---|
| | Tumor size | OCDO | Tumor size | OCDO |
| | (% | (% | (% | (% |
| Molecule | control) | control) | control) | control) |
| Nontreated | 100 | 100 | 100 | 100 |
| control | | | | |
| DDA | 65.4 ± 7 | 14.5 ± 3 | 28.3 ± 8 | 4.5 ± 3 |

It was therefore established in this example that, firstly, the DDA product inhibits the OCDO and, secondly, in vivo, it reduces the tumor size.

### EXAMPLE 11: INHIBITION OF OCDO BY INTRACELLULAR CHOLESTEROL TRANSPORT MODULATORS AND ARYL HYDROCARBON RECEPTOR (AHR RECEPTOR) MODULATORS

Two intracellular cholesterol transport modulators were tested, namely progesterone and U18666A (3-β-(2,20-(diethylamino)ethoxy)androst-5-en-17-one) (see Liscum L et al., J. Biol. Chem., vol. 270 (26) p. 15443-15446, 1995) (lanes 2 and 3, respectively).

Two Ahr receptor (aryl hydrocarbon receptor) modulators, namely 2,3,7,8-tetrachlorodibenzo-p-dioxin (TCDD) and benzo(A)pyrene, were also tested (see lanes 4 and 5, respectively). Finally, two Ahr receptor antagonists were tested, namely resveratrol and 1,3-dichloro-5-[(1E)-2-(4-chorophenyl)ethenyl]-5-benzene (PDM2) (see: “Casper R. F. et al., Mol. Pharmacol., 1999 October; 56(4); 784-90” and “de Medina et al., J. Med Chem., 2005 Jan. 13; 48(1): 287-91)”.

The following experiments were carried out: MCF7 tumor cells were placed in the presence of 0.5 μM of [¹⁴C]CT and were then incubated for 24 hours in the presence of one of the following products:
1. vehicle solvent (0.1% ethanol in a PBS buffer) serving as a control (lane 1)
2. 10 μM of progesterone
3. 10 μM of U18666A
4. 100 nM of TCDD
5. 10 μM of benzo(A)pyrene
6. 10 μM of resveratrol
7. 10 μM of PDM2 (Ant. 1).

Thin-layer chromatographies were carried out and FIG. 24 represents the plate obtained. The OCDO production in the treated cells was quantified from the spots of the various lanes, by applying the protocol given in detail in example 7. The results of this quantification are represented by the histogram of FIG. 24.

The amounts of OCDO produced by MCF7 cells, when they are incubated according to the same protocol as defined above with other Ahr receptor antagonists, was also measured, the control test being brought to 100 and the OCDO production being expressed as % of the control (same control as above). The results are given in the following table:

| | Molecule | OCDO production |
|---|---|---|
| | (concentration | by MCF-7 |
| | 10 μM) | (% control) |
| Control | — | 100 |
| Ahr receptor | Resveratrol | <1 |
| antagonist | Ant 1 (10) | <1 |
| | Ant 2 (10) | <1 |
| | Ant 3 (10) | <1 |
| | Ant 4 (10) | <1 |
| | Ant 5 (10) | <1 |

Ant. 1: (E)-1-(4′-chlorophenyl)-2-(3,5-dichlorophenyl)-ethene;
Ant. 2: (E)-1-(4′-methoxyphenyl)-2-(3,5-fluorophenyl)-ethene;
Ant. 3: (E)-1-(4′-fluorophenyl)-2-(3,5-fluorophenyl)-ethene;
Ant. 4: (E)-1-(4′-trifluoromethylphenyl)-2-(3,5-trifluoromethylphenyl)ethene;
Ant. 5: (E)-1-(4′-fluorophenyl)-2-(3,5-dimethoxy-phenyl)ethene

It therefore appears that the intracellular cholesterol transport modulators and the Ahr receptor antagonists can inhibit OCDO formation and can consequently be used for their anticancer effect.

1-8. (canceled)

9. A method for treating a patient suffering from cancer comprising the steps of:
a) obtaining a tumor cell sample from a patient suffering from cancer;
b) measuring, in said tumor cell sample, the amount of 6-oxo-cholestane-3β, 5α-diol (OCDO);
c) comparing the amount of OCDO measured in step b) with a reference value;
d) providing:
a poor prognosis for said patient if the amount of OCDO compound is significantly enhanced compared with said reference value; or
a good prognosis for said patient if the amount of OCDO compound is lower than said reference value; and
e) treating the patient with an anticancer treatment selected based on the prognosis provided in d).

10. The method for treating a patient suffering from cancer according to claim 9, wherein said method further comprises the steps of:
f) obtaining a second tumor cell sample from the patient suffering from cancer;
g) measuring, in said second tumor cell sample, the amount of 6-oxo-cholestane-3β, 5α-diol (OCDO);
h) comparing the amount of OCDO measured in step g) with that measured in step b);
i) assessing that:
the treatment provided in step e) is effective if the amount of OCDO measured in step g) is lower than that measured in step b); or
the treatment provided in step e) is ineffective if the amount of OCDO measured in step g) is higher than or equal to that measured in step b); and
j) modifying the treatment if it is identified as ineffective in step i).

11. The method for treating a patient suffering from cancer according to claim 9, wherein the tumor cell sample is a liquid extract of the cells of a tumor sample obtained from said patient.

12. A method for treating a patient suffering from cancer comprising the steps of:
a) obtaining a tumor cell sample from a patient suffering from cancer;
b) measuring, in said tumor cell sample, the amount of 6-oxo-cholestane-3β, 5α-diol (OCDO);
c) comparing the amount of OCDO measured in step b) with a reference value;
d) providing:
a good prediction of the therapeutic activity of OCDO inhibitors for said patient if the amount of OCDO compound is significantly enhanced compared with said reference value;
and
e) treating the patient with an OCDO inhibitor when a good prediction of the therapeutic activity is provided in d).

13. The method for treating a patient suffering from cancer according to claim 12, wherein the OCDO inhibitor is selected from:
inhibitors of an enzyme involved in cholesterol biosynthesis, in particular lovastatin, Ro 48 8071, U18666A, AY-9944, triparanol, terbinafine and SKF-525A;
cytochrome P450 inhibitors, lipoxygenases and antioxidants that are active on cholesterol epoxidation, such as ketoconazole and vitamin E;
inhibitors of cholesterol epoxide hydrolase (ChEH) activity, in particular PBPE, PCPE, tesmilifene, dendrogenin A (DDA), tamoxifen, 4 hydroxytamoxifen, raloxifene, nitromiphene, clomiphene, RU 39411, BD-1008, haloperidol, SR 31747A, ibogaine, AC-915, rimcazole, amiodarone, trifluoroperazine, U18666A, AY 9944, triparanol, terbinafine and SKF-525A;
inhibitors selected from the group consisting of:
estrogen receptor antagonists;
anti-estrogen membrane binding site (AEBS) ligands;
ligands of σ-1 and -2 receptors and certain aminoalkyl sterols;
intracellular cholesterol transport inhibitors; and
enzyme inhibitors selected from the group consisting of progesterone and Ahr receptor antagonists.

14. The method for treating a patient suffering from cancer according to claim 13, wherein said OCDO inhibitor is Ro 48-8071 or ibogaine.

15. A method for identifying and treating an patient suffering from cancer comprising the steps of:
a) obtaining a cell sample from a patient;
b) measuring, in said cell sample, the amount of 6-oxo-cholestane-3β, 5α-diol (OCDO);
c) comparing the amount of OCDO measured in step b) with a reference value;
d) assessing that the patient has cancer if the amount of OCDO compound is significantly enhanced compared with said reference value; and
e) treating said patient identified as having cancer at step d) with an anticancer treatment.

16. The method for identifying and treating a patient suffering from cancer according to claim 15, wherein the anticancer treatment consists of administering an effective amount of an OCDO inhibitor to said individual.

17. The method for identifying and treating a patient suffering from cancer according to claim 16, wherein the OCDO inhibitor is selected from:
inhibitors of an enzyme involved in cholesterol biosynthesis, in particular lovastatin, Ro 48 8071, U18666A, AY-9944, triparanol, terbinafine and SKF-525A;
cytochrome P450 inhibitors, lipoxygenases and antioxidants that are active on cholesterol epoxidation, such as ketoconazole and vitamin E;
inhibitors of cholesterol epoxide hydrolase (ChEH) activity, in particular PBPE, PCPE, tesmilifene, dendrogenin A (DDA), tamoxifen, 4 hydroxytamoxifen, raloxifene, nitromiphene, clomiphene, RU 39411, BD-1008, haloperidol, SR 31747A, ibogaine, AC-915, rimcazole, amiodarone, trifluoroperazine, U18666A, AY 9944, triparanol, terbinafine and SKF-525A;
inhibitors selected from the group consisting of:
estrogen receptor antagonists;
anti-estrogen membrane binding site (AEBS) ligands;
ligands of σ-1 and -2 receptors and certain aminoalkyl sterols;
intracellular cholesterol transport inhibitors; and
enzyme inhibitors selected from the group consisting of progesterone and Ahr receptor antagonists.

18. The method for identifying and treating a patient suffering from cancer according to claim 17, wherein said OCDO inhibitor is Ro 48-8071 or ibogaine.

19. The method for identifying and treating a patient suffering from cancer according to claim 15, wherein the cell sample is a liquid extract of the cells of a sample obtained from said patient.
